(19) United States
(12) Patent Application Publication
(10) Pub. No.: US 2022/0389083 A1
(43) Pub. Date: Dec. 8, 2022

(54) PROTEIN-BASED BIOMATERIAL WITH VISCOELASTIC BEHAVIOUR, PROCESS FOR OBTAINING IT AND USES THEREOF

(71) Applicants: Université de Strasbourg, Strasbourg (FR); Centre National De La Recherche Scientifique, Paris (FR); INSERM - Institut National de la Santé et de la Recherche Médicale, Paris (FR)

(72) Inventors: Eya ALOUI, Strasbourg (FR); Marcella DE GIORGI, Strasbourg (FR); Benoît FRISCH, Strasbourg (FR); Philippe LAVALLE, Wintzenheim-Koschersberg (FR); Pierre SCHAAF, Molsheim (FR)

(21) Appl. No.: 17/771,290

(22) PCT Filed: Oct. 23, 2020
(86) PCT No.: PCT/EP2020/079901

§ 371 Date: Apr. 22, 2022

(30) Foreign Application Priority Data
Oct. 25, 2019 (EP) 19306387.2

(51) Int. Cl. C07K 14/765; A61K 47/42; C07K 14/77; A61K 45/06; A61L 27/22; A61L 27/56

(52) U.S. Cl. CPC C07K 14/765; A61K 47/42; C07K 14/77; A61K 45/06; A61L 27/227; A61L 27/56; A61K 38/00

(57) ABSTRACT

The present invention relates to a process of preparation of a biomaterial comprising the steps of:
a) Preparing a solution comprising at least one protein having a solubility in water superior or equal to about 10 mg/mL and at least one salt having solubility in water superior or equal to about 500 mg/mL,
b) Evaporating the solution obtained in step a) as is, as a foam obtained by foaming the solution obtained in step a), or as a mixture thereof, at a temperature comprised of from 4 to 50° C. in atmospheric pressure or at lower temperatures under vacuum or at a pressure lower than atmospheric pressure, until the formation of two non-miscible phases or until obtaining a substantially dry solid, thereby obtaining a biomaterial.

The present invention also relates to a biomaterial obtainable by the process, and to the use of the biomaterial as a support for in vitro tissue engineering and/or for in vitro cell culture and in vitro expansion and/or as an implantable medical device, or as a drug.

### TECHNICAL FIELD

The present invention refers to a process of preparation of a biomaterial, the biomaterial obtainable by the process, as well as the use of the biomaterial as a support for tissue engineering, for cell culture or expansion, as an implantable medical device and as a drug.

Therefore, the present invention has utility in medical fields, notably for tissue engineering, drug delivery, wound dressing and implants.

In the description below, the references into brackets ([ ]) refer to the listing of references situated at the end of the text.

### BACKGROUND OF THE INVENTION

Biomaterials are widely used in various therapeutic applications such as tissue engineering, drug delivery, wound dressings and implants.

Synthetic, living or hybrid, they have gained in a few decades all therapeutic areas: cardiovascular, surgery and orthopedics, dental, ophthalmology, dermatology, urology, nephrology, neurology, endocrinology, especially to regenerate or improve the function of a tissue.

There are several types of biomaterials, but four main categories of biomaterials can be envisaged:
Metals and metal alloys,
Ceramics in the broad sense,
Polymers and soft matter,
Materials of natural origin, such as coral or other constituents extracted from plant or animal organisms, for example chitin, alginate, heparin, fucoidan, cellulose, collagen or fibrin.

This last category of biomaterial is particularly interesting, as materials of natural origin are generally naturally biocompatible, and biodegradable.

Multiple techniques involving heat-aggregation or cross-linking have been used to prepare biomaterials based on materials of natural origin. However, the use of high temperatures and cross-linking agents may lead to irreversible denaturation and alterations of the material's structure, resulting in the loss of its biological properties and possibly revealing new antigenic sites, which can trigger an inflammatory response.

Thus, a need exists of alternative biomaterials based on materials of natural origin that do not have these drawbacks, and that fulfil the needs more natural biomaterials.

### DESCRIPTION OF THE INVENTION

By extensive researches, Applicants have developed new biomaterials exhibiting remarkable mechanical properties and a good stability in water, acidic solutions and cell culture medium.

Surprisingly, the obtained biomaterials display a solid-like behaviour, making it a good candidate as support for tissue engineering or for cell culture.

The present disclosure reports the first biomaterials possibly made entirely of a unique type of protein, or of several chosen proteins that is/are not denatured during the process of preparation of the biomaterial. This implies that the biomaterials of the invention are not likely to trigger an inflammatory response in subjects. Furthermore, the biomaterials are non-cytotoxic and are favourable to cell adhesion and colonization.

As it is made of proteins, the biomaterials of the invention are completely biodegradable.

The process of preparation developed by the Applicants makes it possible to obtain the biomaterial without any chemical or harsh/denaturant formulation conditions. It also allows to modulate the properties of the biomaterials, notably mechanical and intrinsic properties, and to obtain functionalized biomaterials with new properties. Therefore, the Applicants provide versatile biomaterials whose properties can be modulated to adapt to the requirements of targeted therapeutic applications.

Accordingly, in a first aspect, the present invention provides a process of preparation of a biomaterial comprising the steps of:
a) Preparing a solution comprising at least one protein having a solubility in water superior or equal to about 10 mg/mL, and at least one salt having solubility in water superior or equal to about 500 mg/mL,
b) Evaporating the solution obtained in step a) as is, as a foam obtained by foaming the solution obtained in step a), or as a mixture thereof, at a temperature comprised from 4 to 50° C. at atmospheric pressure or at lower temperatures under vacuum or at a pressure lower than atmospheric pressure, until the formation of two non-miscible phases or until obtaining a substantially dry solid, thereby obtaining a biomaterial.

The process of the invention has the significant advantage not to use any covalent cross-linking or heat-aggregation step, thereby allowing obtaining a biomaterial with non-denatured proteins.

The at least one protein used in step a) may be any protein having a solubility in water that is superior or equal to about 10 mg/mL at a temperature of 20° C., for example superior or equal to 20 mg/mL, or to 40 mg/mL, or to 50 mg/mL. For example, the proteins may have a solubility comprised between about 10 mg/mL and 1000 mg/mL. The solubility in water of the protein may be measured by any method known by the person skilled in the art, for example high-performance liquid chromatography (HPLC), attenuated total reflection (ATR)-FTIR spectroscopy, Raman spectroscopy or focused beam reflectance mode (FBRM) measurement. Such proteins may be for example selected among serum proteins such as albumin or globulin, especially γ-globulin. Albumin may be notably selected among human serum albumin, bovine serum albumin, porcine serum albumin, ovalbumin, vegetal albumin, and recombinant albumin, for example recombinant human albumin structurally equivalent to native human serum albumin and produced in rice. Albumin may also be an albumin nanoparticle. As indicated above, “at least one” protein may be used in the process of the invention, meaning that 1, or 2, or 3, or 4, or more different proteins may be used in the solution of step a). Preferably, it may be used between 1 and 3 different proteins. The concentration of the protein in the solution of step a) may be any concentration allowing a mixture with the salt. It can be for example comprised between 10 mg/mL and 500 mg/mL. The man skilled in the art is able to adapt the concentration of protein in the solution depending on the nature of the salt and the salt concentration. However, as explained more in details below, the molar ratio between salt and protein is more important than the protein concentration to obtain a biomaterial with desired features.

The at least one salt in step a) may be any salt having solubility in water superior or equal to about 500 mg/mL at a temperature of 20° C., for example superior or equal to 700 mg/mL, or to 900 mg/mL, or to 1200 mg/mL. For example, the salts may have a solubility comprised between about 500 mg/mL and 3000 mg/mL. This solubility in water of the salt may be measured at a temperature of about 20° C., at a pH of 7, at atmospheric pressure. The solubility in water of the salt may be measured for example by any method known by the person skilled in the art, for example ion chromatography, gas chromatography, acid-base titration, potentiometric titration, volumetry, weighing or Raman spectroscopy. The at least one salt may be for example selected among NaBr, NaI, KI, CaCl₂), MgCl₂, KC₂H₃O₂ and NH₄HCO₂. As indicated above, “at least one” salt may be used in the process of the invention, meaning that 1, or 2, or 3, or 4, or more different salts may be used. Preferably, it may be used between 1 and 3 salts. For example, the at least one salt may comprise NaBr and NaI, NaBr and CaCl₂), KI and CaCl₂), or NaBr/CaCl₂/MgCl₂ for example at molar ratios of 100/100/100, 200/200/200 or 300/300/300. The concentration of the salt in the solution of step a) may be any concentration allowing a mixture with the protein. It can be for example comprised between 0.01 M and 40 M. The man skilled in the art is able to adapt the concentration of salt in the solution depending on the nature of the protein and the protein concentration. However, as explained more in details below, the molar ratio between salt and protein is more important than the salt concentration to obtain a biomaterial with desired features.

Preferably, the at least one protein and the at least one salt may be, in step a), in a molar ratio that is dependent of the nature of the protein and of the nature of the salt for obtaining the biomaterial. Since it has been demonstrated by the Applicants that the formation of the biomaterial is dependent on the paired effect of both concentrations of proteins and salt, the molar ratio salt/protein is a more relevant and reliable parameter to evaluate membrane formation. Knowing this, the molar ratio may be determined by the skilled person in view of his general knowledge and of the desired properties of the insoluble biomaterial, notably its firmness, without undue burden. For example, the molar ratio may be comprised between 100 and 4000, for example between 100 and 3000, or between 300 and 2500, or between 400 and 2000, or between 600 and 1500, or between 650 and 1000, depending on the salt and of the protein used in step a). For example, molar ratio for a mixture of NaBr and albumin may be of 664.

The solution of step a) may be realised by mixing the at least one protein and the at least one salt in an adapted solvent or mixture of solvents, in non-denaturing conditions. The solvent may be chosen by the skilled person in view of his general knowledge and of the nature of the salt and protein, without undue burden. For example, the solvent may be chosen among water, a buffer such as acetate buffer, a mixture of water and buffer and of another water miscible solvent such as ethanol, methanol, acetone, DMF or DMSO. The temperature of the solution during step a) may be of between 5° C. and 40° C.

Step a) can be carried out at any pH avoiding the denaturation of proteins, which is known by the skilled person. Preferably, step a) is performed at a pH comprised between 3.0 and 9.0, the value of 3.0 being optionally excluded. The pH may be for example of between 4.0 and 9.0, or of between 4.0 and 8.0.

The mixture may be realised or may be transferred on any container or support adapted to receive such a mixture. It can be for example a mold of glass or silicone, microscopy or microarray substrates, cell and tissue culture dishes or microwell plates, or a polymeric support around which the biomaterial can take shape. Advantageously, the support may be chosen in view of the desired surface area, shape and thickness of the biomaterial to be obtained. In this purpose, the volume of the mixture to be poured in the container may be chosen depending on the surface area of the support and/or of the desired thickness of the biomaterial. The biomaterial may have any shape, for example a membrane, a full or hollow cylinder, a cone, a sphere, a pavement. For information, the ratio M/S, which is the ratio between the initial weight of protein used for the formulation and the area of the container as exemplified below, may be comprised between 10 mg/cm² and 400 mg/cm², for example between 20 mg/cm² and 400 mg/cm².

Step b) of evaporation may be performed on solution obtained in step a) as in. In this case, step b) is performed directly after step a), or after an intermediate step that does not change the nature or the physical structure of the solution obtained in step a).

Alternatively, step b) may be performed on a foam obtained by foaming the solution obtained in step a). The foam may be obtained, for example, by applying mechanical work on the solution obtained in step a) to increase the surface area of the solution. This can be performed by any method known by the skilled person, for example agitation, dispersing a large volume of gas into the solution obtained at step a), or injecting a gas into the solution obtained at step a).

In another embodiment, step b) may be performed on a mixture of the solution obtained in step a) and of the foam obtained by foaming the solution obtained in step a). In this case, a part of the solution obtained in step a) may be taken and foamed in a separate container. The resulting foam may be evaporated as is or put back with the solution and mixed with it then evaporated as in step b). Preferably, mixing is made gently in order to preserve the foam. The evaporation of the foam produces a highly porous biomaterial.

Evaporating of step b) is made in order to allow the formation of two non-miscible phases or to obtain a substantially dry solid. Advantageously, it is carried out under conditions that make possible to avoid denaturation of the proteins present in the solution, the foam or the mixture thereof. In this purpose, temperature and pressure may be determined, and adjusted relative to each other, to achieve this goal. The person skilled in the art is able to determine these parameters depending on the kind of proteins or salt, and according to his general knowledge. For example, temperature may be comprised from 4 to 50° C. at atmospheric pressure, for example from 4° C. to 20° C., or from 10° C. to 50° C., or from 15° C. to 50° C., or from 20° C. to 50° C., or from 25 to 40° C., or from 25 to 35° C., or from 20 to 30° C. It is also possible to carry out step b) at lower temperatures under vacuum or at a pressure lower than atmospheric pressure. In this case, the temperature may be for example of from 1° C. to 20° C., or from 2° C. to 15° C., or from 5° C. to 10° C., and the pressure may be from 1 to 100 kPa. In any case, evaporating is carried out until the formation of two non-miscible phases or until obtaining a substantially dry solid. This is performed for example when a thin layer salt is deposited on the surface of the biomaterial, or when a solid comprising less than 20% by weight of water, for example less than 10% of water, is obtained. The formation of two non-miscible phases or a substantially dry solid is visually recognisable. For information, it can be optionally verified by measurement of moisture by gravimetric analysis.

The duration of the evaporation stage may be determined by the skilled person without undue burden, according to his general knowledge. This may be function of the kind of proteins, of salt, of the temperature and the pressure chosen for carrying out the process, of the volume of solution to evaporate, or of the shape of the container. For example, evaporation may be performed from 10 hours to 30 days, for example from 1 day to 20 days, or from 2 days to 30 days. A longer duration may be performed, but it is often without any improvement of the technical features of the biomaterial.

Step b) can be carried out at any pH avoiding the denaturation of proteins, which is known by the skilled person. For example, step b) may be performed a pH comprised between 3.0 and 9.0, the value of 3.0 being optionally excluded. The pH may be for example of between 4.0 and 9.0, or of between 4.0 and 8.0, depending on the kind of proteins.

Evaporation may be carried out by any means meeting the criteria listed above, for example an oven or a vacuum oven.

The process of the invention may consist of steps a) and b) as described above, as they allow obtaining a biomaterial of the invention. In this case, the process does not have any other step, and the biomaterial may be obtained directly at the end of step b), as it may be the substantially dry solid, or the solid phase of the two non-miscible phases obtained in step b).

Alternatively, the process of the invention may comprise additional steps allowing obtaining the biomaterial of the invention. Additional steps may be carried out before step a), and/or between steps a) and b), and/or after step b). In this case, the biomaterial may be obtained after the implementation of these additional steps.

For example, the solid phase or the dry solid obtained in step b) may be washed so that at least a part of the salt is eliminated, thereby obtaining the biomaterial. Preferably, the solid phase or the dry solid obtained in step b) may be washed until elimination of at least 90% wt of the at least one salt, thereby obtaining the biomaterial. The washing may be performed until the elimination of, for example, at least 95%, or at least 99% wt of said at least one salt. The washing may be carried out by any means known by the skilled person, for example with distilled water or aqueous buffer. Control of the resulting salt concentration may be performed with any known method, for example by BCA or microanalysis as illustrated below.

A step of soaking of the solid phase or the dry solid obtained in step b) may be carried out, for example after washing. Soaking may allow hydrating the biomaterial. Soaking may be carried out by any means known by the skilled person, for example with distilled water or a buffer, at room temperature for 48 hours.

It is also possible to add at least one additive during step a) and/or step b), or during any additional step as mentioned above. Additive(s) may be any substance allowing modulating as desired the properties of the biomaterial. Additive(s) may also, in some cases, allow removing the salt or part thereof from the biomaterial. Additive(s) may be chosen by the skilled person according to his general knowledge and to the desired properties for the biomaterial. They may be for example selected among polymers, notably non-charged, positively charged, negatively charged and zwitterionic polymers, non-ionic amino acids and particles. Polymer may be any natural polymer chosen among polysaccharides, proteins, peptides and polynucleotides and/or synthetic and semisynthetic polymers. For example, the polymer includes, but is not limited to, polypeptides, homopolypeptides, chitosan, hyaluronic acid, heparin, alginate, chondroitin sulfate, polyarginine, polylysine, ε-polylysine, DEAE dextran, polycyclodextrine, polyallylamine hydrochloride, polyethylenimine, xanthan gum, polyacrylic acid, polyethylene glycol, starch, cellulose and its derivatives, collagen, insulin, fibrinogen, casein, gelatin, gliadin, gluten, elastin, globulin and haemoglobin. The amino acid may be chosen among all suitable amino acids, and preferably among arginine, ornithine, lysine and cysteine. The particle may be any suitable particle, and may be for example selected among nanoparticles, for example carbon nanotubes or graphene, microparticles, bacteria and viral vectors. The additive may be incorporated by any means known by the skilled person. For example, it can be incorporated in the solution obtained in step a) by dissolving and/or suspending said additive directly in the solution obtained in step a), or by dissolving and/or suspending said additive in a water-miscible solvent, then adding the mixture to the solution obtained in step a). Additionally, or alternatively, the additive may be incorporated in the biomaterial obtained in step b) by adsorption of said additive dissolved and/or suspended in a solvent onto the biomaterial obtained in step b). The quantity of additive may be adapted to the proteins and to the desired properties of the biomaterial, and therefore may be determined by the skilled person according to his general knowledge. For example, the percentage of additive may be from 0 to 20% wt, with respect to the total quantity from proteins in the biomaterial, for example from 1 to 18% wt, or from 2 to 15% wt, or from 3 to 12% wt.

In addition or alternatively, at least one active ingredient may be incorporated in the solution obtained in step a) and/or in the biomaterial obtained in step b). This may allow functionalizing the biomaterial. The active ingredient may be incorporated by any means known by the skilled person. For example, it can be incorporated in the solution obtained in step a) by dissolving and/or suspending said active ingredient directly in the solution obtained in step a), or by dissolving and/or suspending said active ingredient in a water-miscible solvent, then adding the mixture to the solution obtained in step a). Additionally or alternatively, the active ingredient may be incorporated in the biomaterial obtained in step b) by adsorption of said active ingredient dissolved and/or suspended in a solvent onto the biomaterial obtained in step b). In this case, the solvent may be selected among water, organic solvents or a mixture of water and a water-miscible solvent. Advantageously, it may be possible to add up to 30% (v/v) of organic solvents to protein/salt solutions without inhibiting membrane formation or altering significantly the properties of the prepared materials, thus allowing their use to incorporate water insoluble active ingredient(s). For example, it may be possible to add up 1%, or 2%, or 3%, or 4%, or 5%, or 6%, or 7%, or 8%, or 9%, or 10%, or 11%, or 12%, or 13%, or 14%, or 15%, or 16%, or 17%, or 18%, or 19% or to 20% or 21% or 22%, or 23%, or 24%, or 25%, or 26%, or 27%, or 28%, or 29% or 30% (v/v) of organic solvents, depending on the nature of protein and solvent. It may be for example up to 15% ethanol, 10% DMSO or 30% acetonitrile or dichloromethane. The skilled person is able to adapt this quantity in function of the protein, the salt and the solvent by performing routine experimentations. Advantageously, it is possible to add up to 30% (v/v) of organic solvents to BSA/salt solutions without inhibiting membrane formation or altering significantly the properties of the prepared materials, thus allowing their use to incorporate water insoluble active substances.

Active ingredient(s) may be any substance allowing confering interesting properties to the biomaterial. Active ingredient(s) may be chosen by the skilled person according to his general knowledge and to the desired properties for the biomaterial. It may be selected among anti-cancer substances, such as goserelin, leuprolide, carmustine, paclitaxel, histrelin or gemcitabine, anti-inflammatory agents, such as diclofenac, immunosuppressants such as azathioprine or methotrexate, immunomodulators such as cyclosporine, modulators of cell-extracellular matrix interaction including cell growth inhibitors such as imatinib or axitinib, anticoagulants such as rivaroxaban or edoxaban, antithrombotic agents such as clopidogrel, enzyme inhibitors, analgesic such as morphine or hydrocodone, antiproliferative agents, antimycotic substances, cytostatic substances, growth factors such as erythropoietin or thrombopoietin, enzymes, hormones, steroids such as hydrocortisone or prednisolone, non-steroidal substances, and anti-histamines such as diphenhydramine or fexofenadine, this list not being limitative. The quantity of active ingredient may be adapted to the proteins and to the desired properties of the biomaterial, and therefore may be determined by the skilled person according to his general knowledge. For example, the percentage of active ingredient may be of from 0 to 30% wt, with respect to the total quantity of proteins in the biomaterial, for example from 0 to 25% wt, or from 1 to 25% wt, or 1 to 20% wt, or from 1 to 18% wt, or from 2 to 15% wt, or from 3 to 12% wt.

In any case, the biomaterial obtained by the process of the invention may contain at least 50% wt of proteins, with respect to the total weight of the biomaterial, for example at least 55%, or at least 60%, or at least 70%, or at least 80%, or at least 90%, or even 100%.

The biomaterial obtained by the implementation of the preparation process is a second object of the invention.

As explained above, the properties of the biomaterial, including its visual appearance, may be modulated by modifying one or more of the parameters of the process of preparation, notably the kind of salt and the ratio salt/protein, thereby offering the possibility to modulate as desired the properties of the biomaterial.

Regarding the firmness of the biomaterial, it may be a solid, insoluble biomaterial, ranging from foam to compact material, including hydrogels.

Regarding the shape of the biomaterial, it may be in any desired shape, depending on the envisaged application and on the container used to prepare the biomaterial. It may be a membrane, a tube, a cylinder, a pad, a ring, this list not being limitative. The biomaterial may also be cut after implementation of the process in order to obtain the desired shape or size.

The biomaterial may be in any desired size, including microparticles, by grinding the biomaterial.

The visual aspect of the biomaterial may be translucent to opaque.

As mentioned above, one of the major advantages of the biomaterial of the invention is that the proteins used to prepare the biomaterial are not denatured during the process of preparation of the invention. The preparation is performed in non-denaturing conditions and the shape and/or the secondary structures of the proteins may be analysed in the solution obtained in step a) and in the biomaterial obtained in step b). “Not denatured” means, according to the invention, that the percentage of secondary structures of said protein in the solution obtained in step a) is at least the same as in a control solution prepared with the corresponding native protein in a similar concentration or as in a control dry native protein powder, and that the percentage of secondary structures of said protein in the biomaterial obtained in step b) presents at least a substantial increase of R-turns and intermolecular R-sheets and a substantial decrease of unordered structures in comparison to the corresponding native protein. The shape and percentage of secondary structures of the protein in the biomaterial may be controlled by any method known by the skilled person, for example by IR analysis or SAXS (Small angle X-ray scattering), as illustrated thereafter.

The biomaterial of the invention is particularly stable over time. Advantageously, it is stable in aqueous solutions, in acidic, neutral and basic pH, and/or in organic solvents such as ethanol, during at least 2 days, and preferably at least 7 days. This means that there is substantially no dissolution of the biomaterial during this period, even if alteration of its structure by breakage of hydrogen bonding and disulfide bridges, and a mass loss lower than 20%, can be observed. For example, the mass loss may be up to 20% in basic solution, and up to 10% in water, ethanol and acidic solutions. Mass loss of the biomaterial may be calculated as illustrated in Example below.

As explained above, the biomaterial may be made only of proteins, notably non-denatured proteins, which imply a great biocompatibility. Advantageously, the biomaterial may be associated with at least one active ingredient as illustrated before in order to improve its biological properties.

Therefore, another object of the invention relates to the use of the biomaterial of the invention as a support for tissue engineering in vitro and/or for in vitro cell culture and expansion and/or an implantable medical device. Indeed, the biomaterials of the invention provide necessary structural and biochemical support for cell growth and as they may be three dimensional, they are particularly suitable for cell culture and drug/cell delivery.

Another object of the invention relates to the use of the biomaterial for use as a drug. As the biomaterial of the invention is able to interact with biological systems, it may for example be used for the constitution of a device for diagnostic purposes, of a tissue or organ substitute or of a device of functional substitution. Therefore, the biomaterial of the invention may be used in vivo as an implantable medical device, notably to replace defective tissues in a subject in need thereof, or for a drug release system. In other words, the invention also describes an implantable medical device comprising the biomaterial of the invention. Advantageously, the device or drug comprising the biomaterial of the invention may be resorbing after a period into a living body, depending on the nature of the biomaterial. For example, the period may be after 20 days after insertion into a living body, or 30 days, or more than 60 days.

This invention is further illustrated by the following examples with regard to the annexed drawings that should not be construed as limiting.

### BRIEF DESCRIPTION OF THE FIGURES

FIG. 1 represents the preparation of albumin-based biomaterials by evaporation in the presence of salt. The molar ratio NaBr/BSA (bovine serum albumin) of the selected formulation is 664. The evaporation is carried on in an oven (37° C.) under atmospheric pressure during 7 days until the biomaterial is completely dry. The excess salt forms a thin layer on the surface of the biomaterial. After evaporation, washing and soaking steps are applied to remove the salt, leaving a water insoluble albumin-based biomaterial.

FIG. 2 represents the tracking of the formation of a BSA/NaBr 664 membrane (evaporation at 37° C., pH=6, M/S=105 mg/cm²) over time. Once washed, the membrane is transparent. The bottom of the mold was covered with a non-stick silicone disk. The irregularity present in the upper right corner is due to an air bubble.

FIG. 3 represents the surface charge titration of albumin in the presence of an increased concentration of NaBr (pH=6).

FIG. 4 represents the rheological properties of albumin-based membranes by an oscillation protocol (frequency ramp: 100 Hz to 0.01 Hz, shear stress controlled: 1 Pa). Elastic component of shear modulus (Pa) is represented by a solid line, viscous component of shear modulus (Pa) is represented by a dashed line.

FIG. 5 represents the stability of BSA/NaBr 664 membranes (evaporation at 37° C. and pH=6) in various dissolution media (from the left to the right: water, saline, NaCl solution 1 M, NaBr solution 1 M, acidic solution pH 3, basic solution pH 10, ethanol and trypsin 0.125 mg/mL). For each dissolution media, membranes were placed in 25 mL of media. The experiments were performed at 37° C. and under stirring during 7 days.

FIG. 6 represents the rheological properties of BSA/NaBr 664 membranes (evaporation at 37° C. and pH=6) incubated in distilled water ( [symbol] ) a solution of 2-mercaptoethanol (2ME, 0.1 M, [symbol] ) and solutions of urea (2 M, [symbol] 4M [symbol] and 8 M [symbol] ). For solution, a batch of 2 [symbol] membranes is placed in 30 mL of media. The experiments were performed at room temperature for 24 h. The rheological properties of the membranes were assessed by an oscillation protocol (frequency ramp: 100 Hz to 0.01 Hz, shear stress controlled: 1 Pa).

FIG. 7 represents (A) the fitting of the amid I band of albumin present in a BSA solution (100 mg/mL, in D₂O) and identification of the subbands of each secondary structure (the fitting curve and the original amid I band spectra are overlapping, residual RMS error <0.005) and (B-E) a comparison of the amid I bands of BSA in (B) a BSA control solution (100 mg/mL, D₂O, solid line) and a BSA/NaBr 664 solution (BSA: 100 mg/mL, NaBr: 1 M, D₂O, dashed line), (C) a BSA control powder (solid line) and a BSA/NaBr 664 membrane (dashed line), (D) a BSA control solution (100 mg/mL, D₂O, solid line) and a BSA control powder (dashed line) and (E) a BSA/NaBr 664 solution (BSA: 100 mg/mL, NaBr: 1M, D₂O, solid line) and a BSA/NaBr 664 membrane (dashed line).

FIG. 8 represents A) scattering curve of BSA solution (solution 2, 40% wt H₂O) and theoretical intensity calculated with CRYSOL using a monomeric BSA protein from 3v03 PBD atomic coordinates (50 harmonics, excluded volume 8.7 10⁴, default values for the other parameters). A scale factor is applied on the theoretical curve to match the data. B) X-ray scattering curves of (a) dry powder of BSA, (b, c) solutions of BSA ((b) solution 1 containing 24.21% wt H₂O, (c) solution 2 containing 39.09% wt H₂O), (d) dry powder of NaBr (* indicate 111, 200 and 220 Bragg reflections), (e) BSA/NaBr 664 membrane. Data are shifted vertically for clarity.

FIG. 9 represents SEM analysis of an albumin-based membrane formulated with a molar ratio NaBr/BSA of 664 under the selected conditions (evaporation at 37° C. and pH=6). The samples were metallized before observation. A) Surface of membrane; B) cross-section of the membrane.

FIG. 10 represents cell viability of Balbc 3T3 fibroblasts treated with membrane extracts (BSA/NaBr 400, BSA/NaBr 664 and BSA/CaCl₂) 700, at 12.5%, 25%, 50% and 100%). The indirect cytotoxicity is estimated by comparing the normalized metabolic activity of Balbc 3T3 cells cultivated during 24 hours in contact with BSA/NaBr 400, BSA/NaBr 664 and BSA/CaCl₂) 700 extracts with the normalized metabolic activity of the positive control (Ctl+). (*) A significant difference was observed between the treated groups and the positive control (Ctl+) (p<0.05). Biological replicates=1, total technical replicates=4.

FIG. 11 represents cell viability of Balbc 3T3 fibroblasts cultivated in direct contact with albumin membranes. The direct cytotoxicity is estimated by comparing the normalized metabolic activity of Balbc 3T3 cells cultivated during 24 hours in contact with BSA/NaBr 400 ( [symbol] ), BSA/NaBr 664 ( [symbol] ) and BSA/CaCl₂) membranes ( [symbol] ) (M/S=25 mg/cm²) with the normalized metabolic activity of the positive control (Ctl+). No significant difference was observed between the Ctl+(□) group and the treated groups. Biological replicates=4, total technical replicates=20.

FIG. 12 represents microscopic examination of Balbc 3T3 mouse fibroblast cultivated during 24 hours in contact with BSA/NaBr membranes. Fibroblasts can be seen around (A) and above (B) the biomaterial.

FIG. 13 represents normalized metabolic activity of Balbc 3T3 fibroblasts measured on albumin membranes (BSA/NaBr 400, BSA/NaBr 664, and BSA/NaCl₂ 700, respectively [symbol] , [symbol] , [symbol] ) freshly transferred to empty unused wells after elimination of the culture media. The cell adhesion is estimated by comparing the normalized metabolic activity of Balbc 3T3 cells between the treated and the untreated groups. (*) A significant difference was observed between the treated groups group and the positive control (Ctl+, □) (p<0.05). (**) A significant difference was observed between the treated groups (p<0.05). Biological replicates=3, total technical replicates=12.

FIG. 14 represents RAW macrophages cultivated during 48 h at 37° C. in contact with BSA membranes (M/S=25 mg/cm², from the left to the right: BSA/NaBr 400, BSA/NaBr 664, BSA/CaCl₂) 700, BSA/NaBr 400 LPS, BSA/NaBr 664 LPS, BSA/CaCl₂) 700 LPS). Nitrite (A) and TNF-α (B) concentrations were measured to assess the activation of macrophages and the inflammatory response. The non-treated group (NT) was cultivated without membranes and without LPS. The LPS was added after 24 h in the culture media (50 ng/mL) in the LPS-treated control group (T LPS) and the LPS-activated groups (LPS). (*) A significant difference was observed between the treated groups and the NT group (p<0.05). (**) A significant difference was observed between the treated groups and the T(LPS) group (p<0.05). Biological replicates=3, total technical replicates=12.

FIG. 15 represents the amplitude sweep tests performed on a hydrated (in water) BSA/NaBr membrane at a fixed frequency of 0.5 Hz, a strain ranging from 0.01 to 100% and at room temperature. A) Storage (G′, Pa) and loss (G″, Pa) modulus are represented as a function of the amplitude (strain, %). B) Loss modulus (G″, Pa) is represented as a function of the amplitude (strain, %). G″ modulus reaches a maximum (Payne effect). C) Storage modulus (G′, Pa) is represented over time (s). Three consecutive amplitude sweep tests were performed.

FIG. 16 represents the evaluation of a cross section of BSA/NaBr membrane using SEM.

FIG. 17 represents FT-IR spectra of amid I band of a heat-treated (80° C., 72 h) BSA/NaBr material (full line) and a control BSA/NaBr material (dotted line).

FIG. 18 represents the production of BSA/NaBr membranes under controlled vacuum (200, 600 and 800 mbars). The control was prepared at atmospheric pressure. A) Visual aspect of the prepared BSA/NaBr membranes. B) Relative yield (%, white), water uptake (%, spotted) and initial expansion (%, hatched) of the prepared BSA/NaBr membranes.

FIG. 19 represents investigation of the effect of organic solvents (Ethanol (%, v/v), DMSO (%, v/v), acetonitrile (%, v/v) and dichloromethane (%, v/v)) incorporated into the BSA/NaBr solution prior to their evaporation at 37° C. on the relative yield (%, white), water uptake (%, spotted) and initial expansion (%, spotted) of BSA/NaBr membranes. The control batches were prepared with 0% solvent (volume ration solvent/solution).

FIG. 20 represents the relative yield (%, white), water uptake (%, spotted) and initial expansion (%, spotted) of albumin membranes prepared with different combinations of the salts CaCl₂) and NaBr. The molar ratio CaCl₂)/BSA was set at 400 and the molar ratio NaBr/BSA was varied from 100 to 1000. The control was prepared with only BSA/CaCl₂).

FIG. 21 represents pre-loading of doxorubicin (DOX) in Albupadmaterials (i.e. the biomaterial according to the invention).

FIG. 22 represents CLSM images of BSA/NaBr and BSA/CaCl₂) membranes pre-loaded with different quantities of doxorubicin (DOX) ranging from 0.25 to 1 mg/membrane (membrane mass=400 mg, thickness≈500 μm). Unloaded membranes (0) were used as a control. The arrows point to the bottom of the samples.

FIG. 23 represents quantification of the doxorubicin (DOX) eliminated during the rinsing process from BSA/NaBr (black) and BSA/CaCl₂) (white) membranes, as a function of initial DOX mass/membrane (μg) of 0, 250, 500, 750 and 1000. A) Mass of DOX eliminated in the rinsing solution (μg). B) Percentage of DOX eliminated during the rinsing process.

FIG. 24 represents Doxorubicin (DOX) release from BSA/NaBr (A μg and B, %) and BSA/CaCl₂ (C μg and D, %)) membranes after 35 days in water at 37° C. Membranes (400 mg) initially loaded with 0.25 mg (cross), 0.5 mg (triangle), 0.75 mg (circle) and 1 mg (solid line) of DOX were tested.

FIG. 25 represents CLSM images of BSA/NaBr and BSA/CaCl₂ membranes (membrane mass=400 mg, thickness=500 μm) pre-loaded with 0.25 mg of FITC-insulin (INS-FITC). Unloaded membranes (0) were used as a control. The arrows point to the bottom of the samples.

FIG. 26 represents FITC-Insulin (INS-FITC) quantification during the rinsing process (A, μg and B, %) and during the release (C, %) from BSA/NaBr (black) and BSA/CaCl₂ (white) membranes after 35 days in water at 37° C. Membranes (400 mg) were initially loaded with 0.25 mg INS-FITC. INS-FITC release. Unloaded membranes (0) were used as control.

FIG. 27 represents visual aspect (A) and water uptake (B) of the implants BSA/NaBr, BSA/CaCl₂, HSA/NaBr, HSA/CaCl₂, and HSA/GLU prepared for in vivo evaluation.

FIG. 28 represents histological evaluation of the materials (BSA/NaBr, BSA/CaCl₂, HSA/NaBr, HSA/CaCl₂, and HSA/GLU) implanted in Nude mice after sacrifice. A) Histological cuts of the implants (marked with an arrow) with their surrounding tissue stained with Gomori staining. B) Representative magnification of histological cuts of an implant (marked with an arrow) with its surrounding tissue stained with Gomori and Picro Sinius staining.

### EXAMPLES

### Example 1: Preparation of Albumin-Based Biomaterials Produced by Evaporation and Salt-Assisted Compaction

Materials

Chemical Reagents.

Bovine serum albumin (fraction V, ≥96%) was purchased from Acros Organics. Human serum albumin (≥96%), Ovalbumin (≥98%) and Gamma-globulins from bovine blood (≥99%) were purchased from Sigma-Aldrich.

Sodium bromide (NaBr), Potassium chloride (KCl) and Potassium acetate (KC₂H₃O₂) were purchased from Sigma-Aldrich. Sodium chloride (NaCl) was purchased from VWR Chemicals. Potassium bromide (KBr) was purchased from Acros Organics. Sodium iodide (NaI) and Dipotassium phosphate (K₂HPO₄) were obtained from Prolabo. Potassium iodide (KI) was purchased from Carbo Erba Reagents. Magnesium chloride (MgCl₂, anhydrous) and Ammonium formate (NH₄HCO₂) were purchased from Fluka. Calcium chloride (CaCl₂, 2H₂O) was purchased from Merck. Potassium carbonate (K₂CO₃) was purchased from Alfa Aesar.

BCA Assay reagents (Bicinchoninic acid solution and Copper(II) sulfate pentahydrate) were purchased from Sigma-Aldrich. Deuterium oxide (D₂O) was purchased from Sigma-Aldrich.

Biological Reagents

Balbc 3T3 mouse fibroblasts (clone A31 ATCC® CCL-163) were cultivated in Dulbecco's Modified Eagle Medium High Glucose (DMEM) containing stabilized glutamine and sodium pyruvate (Dutscher), supplemented with 10% (v/v) of fetal bovine serum (Dutscher) and 1% (v/v) of Penicillin-Streptomycin Solution 100× (final concentrations: 0.06 mg/mL and 0.1 mg/mL respectively) (Dutscher) at 37° C. in 5% CO₂, 95% humidity. Cells were harvested using trypsin (0.5 g/L)-EDTA (0.2 g/L) (Dutscher) for 5 min at 37° C. Thiazolyl Blue Tetrazolium Bromide (MTT) was purchased from Sigma-Aldrich. CellTiter Glo® Viability Assay was purchased from Promega.

RAW 264.7 mouse macrophages (ATCC® TIB-71™) were cultivated in Dulbecco's Modified Eagle Medium High Glucose (DMEM) containing stabilized glutamine (Sigma-Aldrich), supplemented with 5% (v/v) of heat-inactivated fetal bovine serum (Gibco), Penicillin (100 U/mL) (Sigma-Aldrich) and Streptomycin (0.1 mg/mL) (Sigma-Aldrich) at 37° C. in 5% C02, 95% humidity. Cells were harvested using trypsin (0.5 g/L)-EDTA (0.2 g/L) (Sigma-Aldrich) for 5 min at 37° C. Lipopolysaccharide (LPS) from Escherichia coli (K12) was purchased from Invivogen. Purified anti-mouse TNF-α antibody clone 1 F3F3D4 and biotinylated anti-mouse TNF-α antibody clone XT3/XT22 for ELISA testing were purchased from eBioscience/ThermoFisher Scientific. Horseradish Peroxidase Avidin (Avidin HRP) was purchased from Jackson. P-aminobenzenesulfonamide and acetic acid purchased from sigma. N-(1-naphtyl)ethylenediamine dihydrochloride was purchased from Acros Organics.

Methods

Formulation (General Procedure)

A solution of BSA (100 mg/mL) and NaBr 1 M (molar ratio NaBr/BSA=664) in a sodium acetate buffer (0.2 M) at pH 6 was prepared. This solution was deposited in a mold (bottom covered with a non-stick silicone disk) and evaporated at 37° C. for 7 days. The dry biomaterial obtained was washed to remove the salt and soaked in distilled water at room temperature for 48 hours. The water-insoluble membrane (BSA/NaBr 664) was then collected and characterized.

Initial Characterization

The molar ratio salt/albumin (equation 1) and the ratio M/S (equation 2) were used to label the formulations. The relative yield (equation 3), the water absorption (equation 4) and the initial expansion (equation 5) were used to compare the formulated membranes. The density (equation 6) of the material was assessed by immersion of the material in distilled water at room temperature. WBSA represents the initial weight of albumin used for the formulation. Ai represents the area of the container used during the evaporation process. Wd represents the weight of the final dried membrane after washing in distilled water for 48 h and drying in an oven at 37° C. overnight. Vd is the volume of the dried membrane measured by immersion of the material in distilled water at room temperature. Wh represents the weight of the hydrated membrane at equilibrium after immersion in distilled water for 24 h and removal of excess surface water using filter paper. Ah is the area of the hydrated membrane's surface calculated after measuring its diameter using an electronic digital caliper (TACKLIFE-DC01, accuracy ±0.2 mm).

$$Molar ratio salt / albumin = \frac{Molar concentration ( sa}{Molar concentration ( albu} \qquad ( 1 )$$

$$M / S = \frac{{W}_{BSA}}{{A}_{i}} \qquad ( 2 )$$

$$Relative yeild ( Y \% ) = \frac{{W}_{d}}{{W}_{BSA}} ⨯ 100 \qquad ( 3 )$$

$$Water absorption ( W \% ) = \frac{{W}_{h} - {W}_{d}}{{W}_{d}} ⨯ 100 \qquad ( 4 )$$

$$Initial expansion ( E \% ) = \frac{{A}_{h}}{{A}_{i}} ⨯ 100 \qquad ( 5 )$$

$$Density = \frac{{W}_{d}}{{V}_{d}} \qquad ( 6 )$$

Standardized Induced Potential (SIP) Measurements

The titrated solution was prepared by dissolving BSA in mQ water at a concentration of 1 mg/mL. The titration solution was prepared by dissolving NaBr in mQ water at a concentration of 60 mg/mL. The NaBr solution was then used to titrate the protein's surface charge by measuring the induced potential using streaming current detection. A Mütek PCD 02 detector was used. 10 mL of the BSA solution was transferred to the detector tank. Then, after an equilibration time of 5 minutes, consecutive additions of the saline NaBr solution with a frequency of 30 μl/min were performed. The assay was stopped when the measured potential reached a plateau.

BCA Assay

BSA/NaBr 664 membranes (initial BSA concentration=200 mg/mL, M/S=105 mg/cm², n=3) were washed with 4.5 mL of ultrapure water each (2×1.5 mL (2×30 min) then 1×1.5 mL (2 h)). Then, for each rinsing solution, the volume was adjusted to 5 mL using a volumetric flask. Albumin concentration was then determined in the initial solution using in the formulation as well as in the rinsing solution by a BCA test using a standard range (20 μg/mL-1000 μg/mL). The assay was carried out in a 96-well plate. The reagent (bicinchoninic acid/CuSO₄) was added to the solutions (200 μL of reagent to 25 μL of protein solution). Then, the plate was incubated at 37° C. for 30 minutes. The absorbance reading at 560 nm was performed at room temperature using a SAFAS Xenius XM spectrofluorometer (SAFAS Monaco). After calculating the quantity of albumin in the membrane, the quantity of NaBr was deduced.

Microanalysis

Electron-excited X-ray microanalysis was performed on randomly selected areas of the samples using a Quanta™ 250 ESEM (FEI Company, Eindhoven, The Netherlands) operating with an accelerated voltage of electrons of 10 kV (emergence angle=35°, acquisition time=100 s, process time=7.68 μs). Four BSA/NaBr membranes were analysed: BSA/NaBr 400, BSA/NaBr 664, BSA/NaBr 700 and BSA/NaBr 1400 (M/S=113 mg/cm²). The amount of NaBr was estimated using the atomic percentage of brome in the samples.

Rheological Behaviour and Compression Assay

The rheological characterization and the compression assay were performed using a Malvern Kinexus ultra+rheometer equipped with a plane mobile of 2 cm in diameter. Hydrated BSA/NaBr 664 membranes (M/S=105 mg/cm², thickness=1.7 mm) were used. For the oscillation protocol, the samples were subjected to a controlled shear stress of 1 Pa and the measurements were carried out according to an oscillation frequency ramp ranging from 100 Hz to 0.01 Hz at 25° C. For the compression assay, a force ramp of 0.5 N to 40 N (0.04 mm/s) was applied to the samples at 25° C. The elastic modulus (E) of each sample was calculated within the elastic domain of the strain (ε)-stress (σ) curve (equation 7).

σ=e×ε (7)

Traction Assay

The traction assay was performed using an Instron ElectroPuls™ E3000 equipped with a 100 N force sensor. A batch of six hydrated BSA/NaBr 664 membranes (M/S ratio of 110.9 mg/cm²) was used. The samples were cut with a punch to form six specimens with standardized dimensions (initial useful length L₀=40 mm, effective initial width 10=10 mm, initial thickness ε₀=1.74±0.079 mm). The tensile test was then carried out at room temperature with a tensile speed of 0.1 mm/s. The elastic modulus (E) was calculated within the elastic domain of the strain (ε)-stress (σ) curve (equation 7).

Stability in Aqueous Solutions

BSA/NaBr 664 membranes (M/S=105 mg/cm²) were placed, in batches of 3, in 25 mL of the following dissolution media: distilled water, physiological saline (0.9% NaCl), acidic medium (pH 3, colour indicator: bromothymol blue), basic medium (pH 10, colour indicator: bromothymol blue), saline solution of 1 M NaCl, saline solution of 1 M NaBr, ethanol and a trypsin solution (0.125 mg/mL diluted in a PBS buffer). The media containing the membranes was then incubated at 37° C. with shaking (180 rpm) for 7 days. After that, the membranes were washed with water before being characterized for their mass loss (equation 8).

$$Mass loss ( ML \% ) = \frac{Initial weight - Final weight}{Initial weight} ⨯ \qquad ( 8 )$$

IR Analysis

A VERTEX 70 FTIR spectrometer (Bruker, Germany) equipped with a deuterated tryglycine sulphate detector (RT DLaTGS) and a KBr beam-splitter, was employed for infrared measurements. D₂O solutions were used to avoid the spectral overlaps between Amide I band and strong absorption band of water at approximately 1650 cm⁻¹. All samples were placed between two CaF₂ windows. The FTIR spectra of the samples have been recorded at room temperature between 4000 and 800 cm⁻¹ at 2 cm⁻¹ nominal resolution, accumulating 128 scans per spectrum and with a scanning rate of 10 kHz, taking D₂O spectrum as background. The liquid samples (solutions of BSA, BSA/NaBr 400 and BSA/NaBr 664) were prepared in D₂O with a BSA concentration of 100 mg/mL. The solid samples (membranes of BSA/NaBr 400 and BSA/NaBr 664) were hydrated with D₂O.

Spectral analysis was performed by using the spectrometer software OPUS 7.5 (Bruker, Germany). For secondary structure analysis, a curve fitting method was performed for the amide I region (1700-1600 cm⁻¹) of the deconvolved spectra. Prior to curve fitting, the spectra were baseline-corrected for the amide I band using the minima at the low wavenumber (1600 cm⁻¹) and high wavenumber (1700 cm⁻¹) sides. Deconvolution was carried out according to the least-square iterative curve fitting program (Levenberg-Marquardt) using a Gaussian line-shape. The number of subbands and their positions were determined from the deconvolved spectra as well as from the second and fourth derivative of the spectra. For the final fits, in order to reduce the residual RMS error as much as possible (less than 0.005), heights, widths and positions of all bands were adjusted while at least one of these parameters was not allowed to change each time. Finally, second derivatives of the original and the fitted curve were compared to ensure the accuracy of curve fitting. The fractional areas of the fitted components were used to calculate the percentages of different secondary structure elements (a helix, p sheets, p turns, and random coils).

SAXS ICS

Small and wide angle X-ray scattering analysis (SAXS and WAXS) were performed on dry BSA/NaBr 664 membranes as well as on three controls: a dry powder of lyophilized BSA and two solutions of BSA (solution 1: 113.69 mg BSA+36.31 mg H₂O, solution 2: 119.39 mg BSA+79.61 mg H₂O). Measurements were carried out on a Rigaku diffractometer installed on a microfocus rotating anode generator (Micromax™-007 HF) operating at 40 kV and 30 mA. The beam was monochromatized (wavelength λ=1.54 Å) and focused with a confocal Max-Flux Optics™ (Osmics, Inc.) and a three pinholes collimation system. The scattered intensity was measured as a function of the magnitude of the scattering vector q (Equation 9) where 6 is the scattering angle. Two different configurations were used to cover a large scattering vector range.

The low q range was investigated with a 2D multiwires detector located at d=0.81 m from the sample position (0.01 Å⁻¹<q<0.33 Å⁻¹) Higher q values were measured using Fuji imaging plates inserted closer to the sample (d=0.1 m, 0.1 Å⁻¹<q<3 Å⁻¹). Scattering patterns were treated according to the usual procedures for isotropic scattering: intensity was radially integrated and corrected for electronic background, detector efficiency, sample transmission and sample thickness. For BSA solutions, scattering from the pure solvent and the container were also measured and subtracted. Intensities were converted into absolute scale using a calibrated Lupolen standard. The scattering vectors were calibrated using the diffraction peaks of a silver behenate powder.

$$q = \frac{4 π}{λ} sin ( \frac{θ}{2} ) \qquad ( 9 )$$

Scanning Electron Microscopy (SEM)

Scanning electron microscopy assessments were performed using a Quanta™ 250 ESEM (FEI Company, Eidhoven, The Netherlands) operating with an accelerated voltage of electrons of 10 kV. BSA/NaBr 664 membranes (M/S ratio=105 mg/cm²) were dried at 37° C. during 48 hours. The samples were then coated with a gold-palladium alloy using a Hummer Jr sputtering device (Technics, Union City, Calif., USA). The surface and the cross section were examined.

Extract Cytotoxicity Test

For the indirect cytotoxicity assessment, the ISO standard (ISO 10993-5 (2009)) was followed. The membranes used during this test were BSA/NaBr 400, BSA/NaBr 664 and BSA/CaCl₂ 700. The membranes were formulated with a ratio M/S of 25 mg/cm². The membranes were washed with ethanol 70% then with sterile PBS 1× and sterilized for 15 minutes under UV light. Then, they were stored in sterile PBS 1× until further use. Each membrane was transferred to a 12-well plate and extracted in 1.5 mL of culture medium (DMEM+FBS (10%)+PS (1%)) under stirring at 37° C. during 72 h. Dilutions containing 12.5%, 25%, 50% and 100% (v/v) of the extracts were then prepared. Balbc 3T3 mouse fibroblasts (clone A31 ATCC® CCL-163) were cultivated in a 96-well plate at 8000 cells per well (culture medium: DMEM+FBS (10%)+PS (1%)) at 37° C. for 24 h. The following day, the culture medium in each well was replaced with 100 μL of the diluted extracts. A positive and a negative control were prepared with only the culture media and with the culture media containing 20% of DMSO respectively. The plate was then incubated at 37° C. for 24 h. After incubation, the culture medium in each well was replaced with 100 μL of a solution of MTT diluted in fresh culture medium (1 mg/mL) and the plate was incubated at 37° C. for 2 h. The formazan crystals were then solubilized in 80 μL of DMSO and the absorbance at 560 nm was measured using the SAFAS apparatus after an equilibration of 15 min at room temperature. The metabolic activity of the positive control was used to determine the percentage of viable cells in each group.

Direct Cytotoxicity Test

The membranes used during this test were BSA/NaBr 400, BSA/NaBr 664 and BSA/CaCl₂ 700. The membranes were formulated with a ratio M/S of 25 mg/cm² in non-stick silicone molds. The hydrated membranes were cut using a circular punch to obtain small disks (diameter=5 mm, thickness=0.7 mm). The disks were washed with ethanol 70% then with sterile PBS 1× and sterilized for 15 minutes under UV light. Then, they were stored in sterile PBS 1× until further use. For the direct cytotoxicity assessment, the sterilized disks were transferred to a black-walled 96-well plate. Balbc 3T3 mouse fibroblasts (clone A31 ATCC® CCL-163) were then added to the plate at 8000 cells per well (culture medium: DMEM+FBS (10%)+PS (1%)) directly on the disks of biomaterials. A positive and a negative control were added with only the culture media and with the culture media containing 20% of DMSO respectively. The plate was then incubated at 37° C. for 24 h. After incubation, the plate was equilibrated at room temperature for 30 minutes. The culture media was eliminated. In each well, 50 μL of new culture media was added followed by 50 μL of the CellTiter-Glo® reagent. The bioluminescence was then measured using the SAFAS apparatus and the following protocol: the plate was stirred for 2 minutes then left to equilibrate for 10 minutes before measuring the bioluminescence. The metabolic activity of the positive control was used to determine the percentage of viable cells in each group.

Macrophage Activation Assay

The membranes used during this test were BSA/NaBr 400, BSA/NaBr 664 and BSA/CaCl₂ 700. The membranes were formulated with a ratio M/S of 25 mg/cm² in non-stick silicone molds. The hydrated membranes were cut using a circular punch to obtain small disks (diameter=5 mm, thickness=0.7 mm). The disks were washed with ethanol 70% then with sterile PBS 1× and sterilized for 15 minutes under UV light. Then, they were stored in sterile PBS 1× until further use. For the macrophage activation assay, the sterilized disks were transferred to a 96-well plate. RAW 264.7 macrophages were then added to the plate at 100000 cells per well (culture medium: DMEM+FBS (5%)+PS (1%)) directly on the disks of biomaterials. After 24 h of incubation at 37° C., LPS was added to the LPS-treated groups to obtain a final concentration of 50 ng/mL in each well. The plate is then incubated for another 24 h at 37° C. A negative and a positive control were prepared with only the culture media and with the culture media containing 50 ng/mL of LPS respectively. The shapes of the cells were then evaluated by microscopy and NO and TNF-α production were assessed as follows.

Assessment of NO Production

Concentrations of nitrite in cell supernatants were evaluated by Griess test (n=3). 60 μL of Griess reagent (v/v mixture of 58.1 mM p-aminobenzene sulfonamide in 30% acetic acid and 3.9 mM N-(1-naphtyl)ethylenediamine dihydrochloride in 60% acetic acid) were added to 40 μL of supernatant and the absorbance at 543 nm was measured and compared to a sodium nitrite standard curve.

Assessment of TNF-α Production

TNF-α concentration in cell supernatants was evaluated by ELISA (n=3) using commercially available reagents and following the manufacturer instructions. Capture antibody was diluted to 1 μg/mL in a 0.05 M pH 9.6 carbonate/bicarbonate buffer and coated 1 night at 4° C. before blocking with PBS 0.05% Tween 20 1% BSA (1 h, 37° C.). Samples were then diluted with culture media and incubated with capture antibody (2 h, 37° C.) before detection antibody diluted to 0.5 μg/mL in PBS 0.05% Tween 20 1% BSA was added (1 h, 37° C.). Avidin HRP was then introduced (45 min, 37° C.) and revelation was conducted by adding a solution of 1.25 mM tetramethylbenzidine and 13.05 mM H₂O₂ in 0.1 M pH 5 citrate buffer. Revelation was finally stopped by addition of 1 M HCl and absorbance was measured at 450 nm.

Statistical Analysis

Data were analysed by using R (version 3.6.1, R Foundation for Statistical Computing, Vienna, Austria). The normality of distribution was determined with the Shapiro-Wilk test. The equality of variances was determined with the F-test. When the data were normally distributed and the variances of the samples were equal, a t-test (2-tailed) was used to compare two means. When these two conditions did not apply, a Mann-Whitney test was performed instead. Values were considered statistically significant at p<0.05.

Results and Discussion

Formulation Parameters Screening

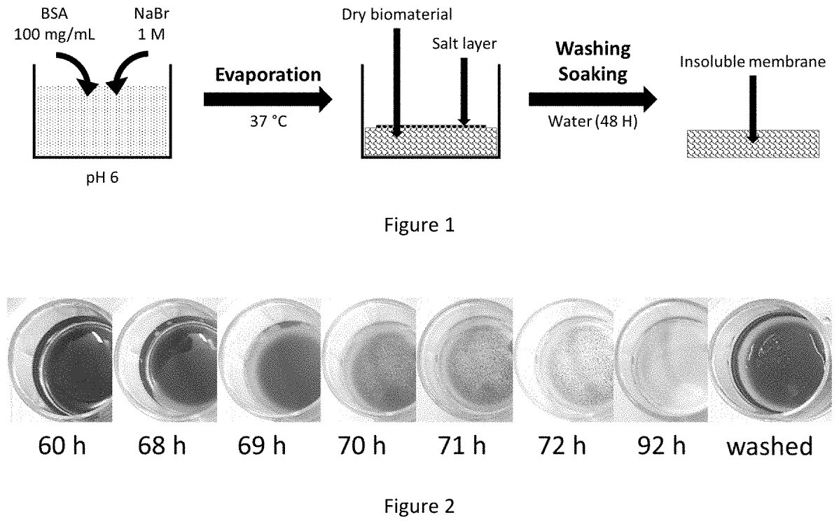

The solubility of albumin in aqueous solutions and its thermal and pH-stability regions were thoroughly studied. Previous studies showed that albumin is stable in a range of pH between 3 and 9. It was also shown that the denaturation temperature is dependent on the pH and decreases at low pH values (62° C. at pH 7.4, 46.8° C. at pH 3.5). In this work, the evaporation temperature was set to 37° C. and the pH to 6 in order to preserve as much as possible the native structure of albumin. Then, a thorough screening of operational conditions was carried out to identify those allowing the formation of interesting biomaterials. Albumin solutions (protein alone or with salt) formulated at controlled pH (pH=6) were evaporated in an oven at 37° C. until the residues were completely dry (see FIG. 1). Afterwards, the residues were washed to eliminate the excess salt and protein, then soaked in distilled water for 48 hours to assess their water solubility. Only the formulations that produced water insoluble materials were selected. For potential applications in biomaterial fields, water insoluble membranes exhibiting good handleability are the most promising.

For a material formulated with M/S of 105 mg/cm², a solid material was obtained after 68 to 69 hours (see FIG. 2). After 69 hours, a thin white layer of excess salt forms on the surface of the material as the residual water evaporates. After washing, the layer of salt is rapidly eliminated and a translucent membrane is obtained (see FIG. 2). An evaporation duration of 7 days was found to be optimal for membranes with M/S of 105 mg/cm² in order to allow the formation and the compaction of the material.

First Parameter: Salt

First, the solubility of albumin residues prepared without salt was verified, confirming the importance of salt in the formulation of albumin membranes. Then, a thorough screening of many salts at three different concentrations (0.5 M, 1 M and 2 M) was performed. As described previously, the water solubility of the dry residues after 48 hours in distilled water was used to identify the salts that allow membrane formation.

According to Hofmeister series, different ions have different effects on protein stability and solubility. The lyotropic effect is related to the size, the charge density and polarizability of the ions. When working with solutions containing high concentrations of salt and protein, the implication of the interaction between the solvent molecules, the salt's ions and the protein should be considered. During this experiment, 12 salts with various anions and cations couples were tested: KCl, KBr, KI, NaCl, NaBr, NaI, CaCl₂, MgCl₂, KC₂H₃O₂, NH₄HCO₂, K₂CO₃ and K₂HPO₄. The results of this experiment suggest that the formation of albumin membranes is dependent on both the type of salt and its concentration. In the presence of KCl, NaCl or KBr, albumin molecules do not organize into membranes, the dry residue is a mixture of crystallized salt and dry protein which are entirely soluble in water. Additionally, the premature aggregation of albumin in the initial solutions prepared with salts containing divalent anions (K₂CO₃ and K₂HPO₄) prevents membrane formation. Water insoluble membranes were obtained with 7 of the 12 salts: NaBr, KI, NaI, CaCl₂, MgCl₂, KC₂H₃O₂ and NH₄HCO₂. With NaBr, membranes were obtained with all three tested concentrations. Although, with the other 6 salts, albumin membranes were obtained only with certain concentrations. The physical aspect and the properties of these membranes (water absorption, initial expansion, handleability) vary considerably depending on the type of salt used and its concentration.

BSA/NaBr 664 membranes (initial salt concentration=1 M) exhibited good mechanical strength and handleability. These membranes (M/S=105 mg/cm²) were produced with a relative yield of 87.6%±4.1%, their water absorption and initial expansion were estimated at 123.1%±6.8% and 121.6%±4.7% respectively (n=8) and their density was 1.29±0.02 g/cm³. This formulation was selected as a reference for the identification of the parameters for albumin-based membranes formation and for the characterization of these materials.

Second Parameter: Salt/Albumin Molar Ratio

It was shown previously that the salt concentration had an impact on membrane formation. However, it is unclear whether the concentration of salt should be considered as an independent parameter or it should be paired with the concentration of albumin in a given solution. The effect of the initial concentrations of BSA and NaBr on the formation of membranes was assessed by comparing the membranes obtained during two assays: the first assay involved a constant concentration of NaBr and a variable concentration of BSA, therefore a variable molar ratio salt/albumin, while the second assay required both concentrations to be modified at the same time without changing the molar ratio salt/albumin. In the first assay, solutions of 100 mg/mL, 200 mg/mL, 300 mg/mL and 400 mg/mL of BSA were prepared with 1 M NaBr. The molar ratios NaBr/BSA were respectively 664, 332, 221 and 166. Decreasing the molar ratio resulted in a notable decrease of the formulation yield (respectively 86.4%, 77.2%, 62.7% and 0%) and the obtained membranes vary considerably in terms of visual aspect and water absorption. Furthermore, the residue obtained after the evaporation of the solution with the concentration of albumin of 400 mg/mL (molar ratio salt/albumin=166) was entirely water soluble. In the second assay, the prepared solutions contained the following concentrations of BSA and NaBr respectively: 100 mg/mL with 1 M, 200 mg/mL with 2 M, 300 mg/mL with 3 M and 400 mg/mL with 4 M. The molar ratio NaBr/BSA was 664 in all solutions. Unlike the first assay, all the solutions of the second assay led to membrane formation. The obtained membranes share the same visual aspect and exhibit similar properties. Therefore, the effect of salt concentration on membrane formation cannot be assessed without pairing it with the concentration of albumin in the initial solution. Since the formation of membranes is dependent on the paired effect of both concentrations of salt and albumin, the molar ratio salt/albumin proves to be a more relevant and reliable parameter to evaluate albumin membrane formation.

The next step was to identify the range of molar ratios NaBr/BSA in which membranes can be formed. BSA solutions were prepared with a set concentration of albumin (100 mg/m) and molar ratios NaBr/BSA from 50 to 2000. These solutions were evaporated and the resulting materials were soaked in water for 48 hours as described earlier. Fully formed membranes were obtained within the range of molar ratios 100 and 3000, particularly within 200 to 2000. For the lowest and the highest molar ratios of this range, the obtained membranes are less robust and more prone to breakage and degradation during handling, but they are however acceptable.

Influence of Albumin Surface Charge

Because of the well-established implication of the ionic content of the initial solution on the formulation of albumin membranes, albumin's surface charge should be evaluated to provide a better understanding of the ionic phenomenon leading to membrane formation. Albumin's surface charge is dependent on the pH of the initial solution and its ionic strength.

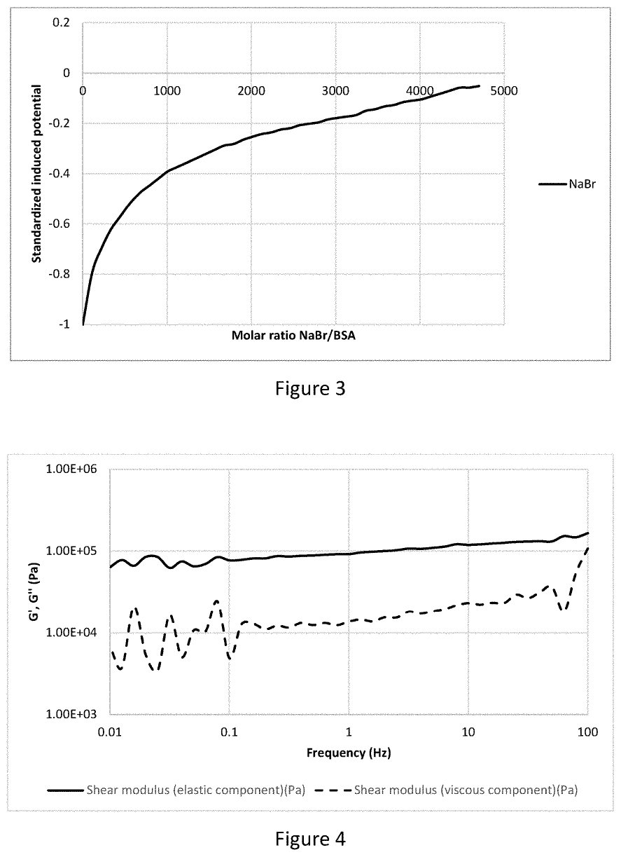

To evaluate the influence of pH on the formation of membranes, solutions of albumin and NaBr at a molar ratio NaBr/BSA of 664 were prepared at pH values of 4, 5, 6, 7 and 8. With an isoelectric point of 4.7 and an isoionic point of 5.2, BSA has a net negative charge at pH 6, 7 and 8, a net positive charge at pH 4, and is a zwitterion around pH 5. Albumin membranes were obtained at all tested pH values. These membranes shared similar visual aspect and formulation yields but their water absorption and initial expansion were significantly different. The membranes formulated at the highest pH values have higher water absorptions and initial expansions. Therefore, pH seems to have a moderate influence on the formation of membranes. Furthermore, the study of albumin surface charge at pH 6 revealed that by adding salt, the global surface charge of the protein increases due to the interactions between the protein and the cations of the salt. In fact, the measured induced potential increases greatly by increasing the salt concentration before reaching a plateau (see FIG. 3). The increase of albumin surface charge can result in the reduction of electrostatic repulsions between the molecules and promoting their agglomeration to form albumin membranes.

Residual Salt Content

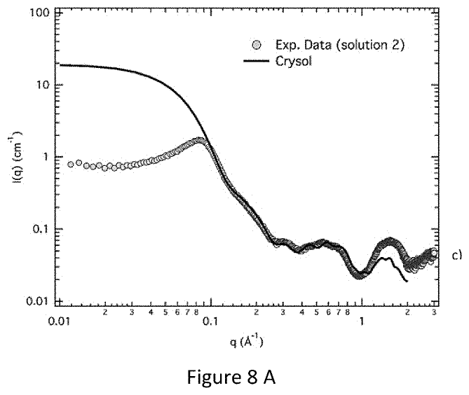

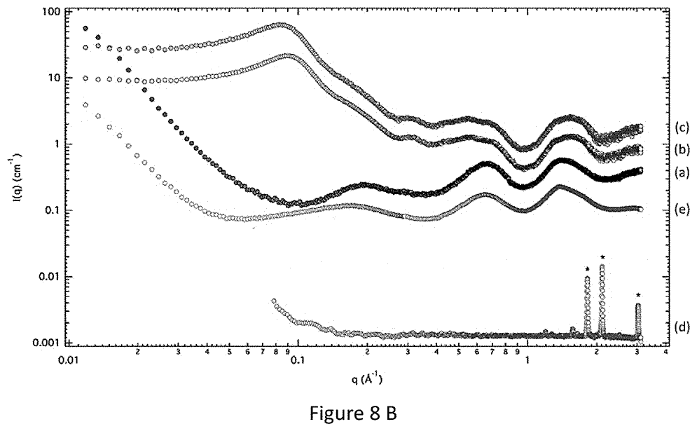

In anticipation of biological evaluation, the final composition and the residual salt content in the formulated materials should be well characterized. Therefore, to determine the final composition of the BSA/NaBr 664 membranes and quantify the residual NaBr, two complementary methods were used. First, using a BCA assay, albumin was quantified in the rinsing water used for washing the BSA/NaBr 664 membranes. After evaporation of the rinsing solution, the dry residue was weighed and the quantity of NaBr eliminated by the washing process was calculated and compared to the quantity of NaBr used initially to formulate the membranes. The residual NaBr content in the washed BSA/NaBr 664 membrane was estimated at less than 1% (% wt). To verify these results and target specifically the bromine content, the final composition of BSA/NaBr 664 membranes was directly analysed by microanalysis. Microanalysis was performed directly on the dried membranes and revealed that the bromine content in the tested membranes was not detectable in the analysed area (analyzed area=1180 μm², sample thickness=1 mm) Furthermore, the analysis of the BSA/NaBr membranes by X-ray diffraction (see FIG. 8) did not detect any traces of crystalline NaBr in the membranes. In conclusion, the NaBr added initially in the solution for the formulation of albumin-based biomaterials is eliminated during the washing process.

Mechanical Properties

The viscoelastic behaviour of the selected albumin-based biomaterial was studied after its saturation with water. The conservation modulus (G′) was found to be higher than the loss modulus (G″) (see FIG. 4). Furthermore, no sol-gel transition was observed. Therefore, within the range of frequency tested, the membranes display a solid-like behaviour.

During the compression assay, it was found that the membrane formulated with BSA and NaBr with a molar ratio of 664 has an elastic modulus of 0.7 MPa. Furthermore, tensile tests were carried out on a batch of albumin membranes (n=6) formulated by the same way. The elastic modulus of the membranes calculated after the traction assay is 0.87±0.12 MPa. Therefore, both assays give similar results. The maximum stress that caused the breakage of the biomaterial was estimated at 0.19±0.03 MPa.

Table 1 below shows the comparison between traction and compression results. The membranes used were formulated with BSA (bovine serum albumin) and NaBr at a molar ration NaBr/BSA of 664 under the selected conditions (evaporation at 37° C. and pH=6). The tests were performed on hydrated biomaterials.

TABLE 1

| | Traction | Compression |
|---|---|---|
| | assay | assay |
| Initial thickness | 1.74 ± 0.08 | 1.7 |
| (mm) | | |
| Strain to | 26.2 ± 4.7 | No rupture |
| rupture (%) | | |
| Maximal stress | 0.19 ± 0.03 | >0.13 |
| (MPa) | | |
| Maximal force | 3.2 ± 0.5 | >40 |
| (N) | | |
| Elastic | 0.86 ± 0.13 | 0.7 |
| modulus (MPa) | | |

Stability in Aqueous Solutions

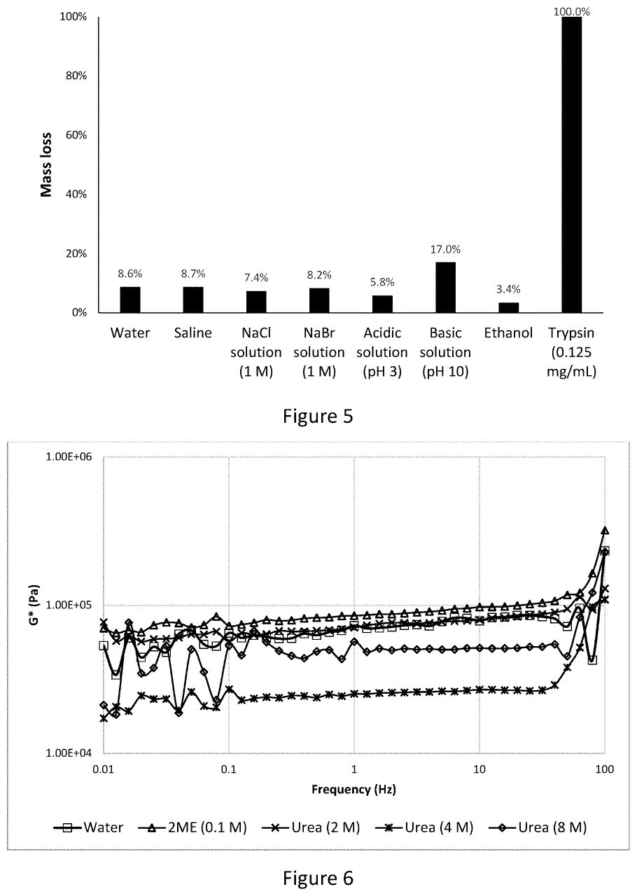

It is important for a biomaterial to be used in contact with biological fluids to have a good stability in aqueous media. For applications such as the formulation of implants or scaffolds for tissue regeneration, the biomaterial should be insoluble in water or have a very slow degradation process. Therefore, the stability in aqueous solutions of the membrane BSA/NaBr 664 was tested. The biodegradability of the membrane was also tested in a trypsin solution (see FIG. 5).

The membranes incubated in contact with the trypsin solution were completely degraded and dissolved in the buffer. As for the membranes incubated for 7 days at 37° C. without the protease, each batch of membranes lost less than 10% of its initial mass in each dissolution media, with the exception of membranes incubated in the basic solution, which lost 17% of their mass. Furthermore, the formulated albumin-based membranes are insoluble in an aqueous medium and remain intact for more than 7 days of incubation in those media at 37° C. In fact, these membranes could be stored up to a month in distilled water without showing any degradation. In addition, these biomaterials show resistance to acidic and basic pH. The mass loss observed in water and ethanol could be explained mostly by the erosion of the membrane caused by frictions against the sides of the tubes during its agitation. Therefore, these albumin-based membranes are very stable in aqueous solutions and are completely biodegradable.

The stability of BSA/NaBr 664 membranes in solutions of urea (2 M, 4 M and 8 M) and 2-mercaptoethanol (0.1 M) was also tested. Urea and 2-mercaptoethanol are well-described denaturants that can induce protein unfolding by breaking hydrogen bonds and disulfide bridges respectively. Even though the tested albumin-based membranes did not break or dissolve, the diameter of the hydrated membranes (Ah) increased significantly, indicating an increase of their initial expansion (E %), as their complex modulus (G*) decreased, indicating a decrease of their elastic modulus (E) (see FIG. 6). Furthermore, the breakage of hydrogen bonding and disulfide bridges altered the structure of the membranes but did not allow their dissolution.

Evaluation of the Conformation of Albumin

Ir Analysis.

Fourier transform infrared spectroscopy (FTIR) is a well-established method used to assess the secondary structures of proteins. The infrared spectra of proteins are characterised by a set of absorption regions in the absorption spectrum known respectively as the amide region and the CH region. Information about the secondary structures can be obtained from the spectra primarily from the amide I region (1700-1600 cm⁻¹) and the amide II region (1600-1500 cm⁻¹). The amide I region reflects mainly the C═O stretching vibration of the peptide group, which gives information on the proteins secondary structures. Furthermore, this technique allows the exploration of the secondary structures of proteins contained in highly concentrated solutions and in solid materials and can either be used with hydrated or dry materials. FTIR was used to evaluate the structure of albumin in BSA/NaBr membranes. In this experiment, to prevent the H₂O bands from interfering with the Amid I band located in the same absorption range, D₂O was used to hydrate a readily available BSA/NaBr 664 membrane and to prepare two control solutions: a solution of BSA (100 mg/mL) and a solution of BSA and NaBr (BSA concentration=100 mg/mL, BSA/NaBr ratio=1:664) comparable to the one used to formulate the BSA/NaBr 664 membranes.

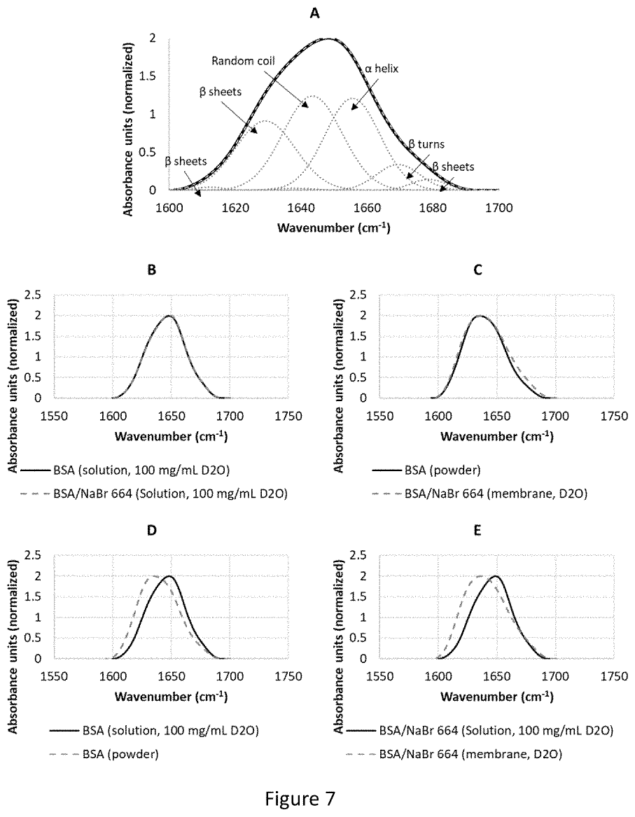

The analysis of the deconvoluted spectra of the amid I band revealed the existence of multiple subbands that were identified using the data already available in the scientific literature. Six subbands were found in the amide I band of the tested samples: α-helix (1655 cm⁻¹), β-sheets (1612, 1629 and 1678 cm⁻¹), β-turns (1669 cm⁻¹) and random coils (1643 cm⁻¹) subbands (see FIG. 7). The percentage of these secondary structures was then calculated (residual RMS error <0.005). Very similar percentages of β-sheets, β-turns, α-helix and random coils were obtained for the albumin present in the solutions of BSA and BSA/NaBr 664. Therefore, in the established experimental conditions, NaBr does not alter the secondary structure of albumin. However, in the BSA/NaBr 664 membrane, there seems to be an increase in β-sheets and β-turns and a decrease in α-helix and random coils (Table 2). The unfolding of proteins is characterized by an increase in random coils which are inorganized secondary structures. In the formulation of the membranes, the albumin seems to some of these inorganized structures in favour of β-organized structures.

Table 2 below shows the analysis of the percentage of each secondary structure identified in the amid I band of albumin present in a BSA solution (100 mg/mL, in D₂O), a BSA/NaBr 664 solution (BSA concentration=100 mg/mL, in D₂O) and a BSA/NaBr 664 membrane (residual RMS error <0.005). A BSA solution (BSA 100 mg/mL 80° C. D₂O) was incubated overnight at 80° C. and was used as a reference for denatured protein.

TABLE 2

| | α | β | β | Random | Intermolecular |
|---|---|---|---|---|---|
| | helix | sheets | turns | coil | β sheets |
| BSA/NaBr 664 | 31.7% | 28.5% | 6.0% | 33.8% | 3.8% |
| solution D₂O | | | | | |
| BSA/NaBr 664 | 21.8% | 46.8% | 16.5% | 14.8% | 23.6% |
| membrane D₂O | | | | | |
| BSA 100 mg/mL | 30.6% | 28.2% | 6.9% | 34.3% | 2.9% |
| D₂O | | | | | |
| BSA 100 mg/mL | 8.8% | 48.8% | 5.7% | 36.7% | 2.6% |
| 80° C. D₂O | | | | | |

Crystallography SAXS ICS

Small and wide-angle X-ray scattering measurements (SAXS, WAXS) probe the inter-atomic distances within the sample. For proteins in solution, they reveal the hierarchical structural levels of the macromolecules. Distinct scattering vector domains are therefore linked to characteristic organizations of the molecules, such as the quaternary and tertiary structures (shape and size of the molecules, q<≈0.2 Å⁻¹), the inter-domains correlations (=0.2<q<≈0.5 Å⁻¹), the intra-domain organization (≈0.5<q<≈0.8 Å⁻¹) and the secondary structure (≈1.1<q<≈1.9 Å⁻¹). WAXS is very sensitive to structural disorder or fluctuations and is able to evidence small changes in macromolecular organization. The interpretation of the data is however very complex and often needs comparisons with theoretical calculations based on crystallographic results. In dilute regime where inter-molecular correlations are absent, scattering measurements only reflect the intra-molecular properties (form factor). When the concentration is increased or in bulk membrane properties this is no longer the case and inter-molecular correlations deeply contribute to the scattered intensity.

The structural organization of BSA/NaBr 664 membranes was probed by SAXS and WAXS. Washed and dried membranes were measured (e, FIG. 2.S8B) and compared to native BSA (a, FIG. 2.S8B). Two BSA solutions (b and c, FIG. 2.S8B) prepared at different concentrations (solution 1 24.2% wt H₂O, solution 2 40% wt H₂O) were also considered to provide a reference for the interpretation of the scattering patterns. The experimental intensity is presented in FIG. 2.S8A (solution 2) and compared to the theoretical curve calculated with CRYSOL and based on a crystallographic description of a monomeric BSA (PDB atomic coordinates 3v03). In between 0.1 and 1 Å⁻¹, both experimental and theoretical curves correspond well. Small deviations certainly reflect the dynamical fluctuations and disorder within the proteins in solution. Below 0.1 Å⁻¹ a peak is observed around 0.084 Å⁻¹. It is related to the spatial correlations between BSA resulting from the electrostatic repulsion which is not considered in the calculations. By increasing the protein concentration (solution 1), this peak is shifted to higher q values (0.09 Å⁻¹) due to a reduction of the distance between the centers of mass of the molecules. At high q values (>1 Å⁻¹), a large maximum was observed around 1.5 Å⁻¹. In this region the model is less accurate but this is out of the acceptable q range for the program (˜0.5 Å⁻¹). CRYSOL is therefore able to explain the main characteristics of the data for BSA in solution. Since the protein mainly contains α-helix in the crystalline ordered state (as used in the calculation), the predicted shoulder observed around 0.2 Å⁻¹ (d=2π/q˜30 Å) is probably associated to the correlations between α-domains while the three contributions in between 0.4 and 0.7 Å⁻¹ (9→15 Å) are related to the internal structure of the domains and more specifically to the α-helix packing. The broad maximum around 1.5 Å⁻¹ (˜4 Å) not correctly described theoretically is found for all proteins and is less sensitive to the main characteristic of the internal organization (α-helix or β-sheets).

The scattering pattern of lyophilized powder BSA is presented is FIG. 2.S8B (a). This amorphous sample is used as a reference for unsolvated native BSA. The intensity is highly modified compared to simple BSA solutions. The data interpretation is more complex since intra and inter-molecular correlations now contribute to the scattered intensity. Disorder, inter-molecular correlations or specific rearrangements may explain potential modifications of the scattering curves between the solution and the solid state. It is no longer possible to determine the shape and size of the proteins, only shorter length scales associated to the internal structure or inter-molecular correlations are measurable. First a large upturn was observed at very low q. This behavior is not related to any particular organization and is simply a consequence of the powder nature of the sample (Porod scattering). In the absence of such contribution the intensity would tend to a constant low value (q→0). In the range 0.1→0.3 Å⁻¹, a small maximum is observed around 0.19 Å⁻¹ (33 Å). This peak could be related to the high concentration limit of the correlation peak observed for “spherical” BSA molecules in solution. However this seems unlikely since the average distance between proteins in contact (33 Å) would be very close to their radius of gyration (Rg=28.7±1.5 Å). This maximum is more likely associated to the correlations between protein domains (including intra and inter-molecular contributions) and is equivalent to the shoulder observed previously around 0.2 Å⁻¹ in BSA solutions (pure intra-molecular contribution). α-domains are therefore still present in the solid state. Other modifications are observed in between 0.3 and 1 Å⁻¹. The structured massif in BSA solutions now reduces to a single contribution close to 0.66 Å⁻¹. This maximum indicates a single average distance associated to the α-helix in the BSA domains with a characteristic packing distance of 9.5 Å (a classical value for this kind of organization). Finally a large contribution was still observed around 1.42 Å⁻¹. In comparison to BSA solutions, the width of the maximum is reduced and its position is shifted to smaller q values. This observation may be linked to the increase of β-sheets content in the solid BSA which are characterized by a 4.7 Å strand-to-strand distance. The scattering pattern of powder BSA therefore evidence slight modifications of the internal structure in the disordered solid state.

The scattering curves of BSA/NaBr 664 membranes are presented in FIG. 2.S8B (e). The upturn observed below 0.04 Å⁻¹ is related the porous nature of the membranes (Porod scattering). No Bragg peaks are visible indicating the absence of crystalline NaBr in the membranes (pure NaBr salt diffractogram is presented in FIG. 2.S8B (d) for comparison). Only small but significant modifications are observed in comparison with pure amorphous BSA powder. The first peak associated to inter-domains correlations is shifted to lower q value (from 0.19 Å⁻¹ to 0.17 Å⁻¹) indicating a larger distance (from 33.1 Å to 37.9 Å) between the protein domains (intra and inter contributions). This behavior is difficult to interpret but results from small variation of the tertiary structure such as a reduction of the α-domains. The peak around 0.65 Å⁻¹ associated to α-helix packing is still present but less intense than for pure BSA. This is consistent with a modification of the helix organization in the membranes. The shape of the large maximum around 1.4 Å⁻¹ is also modified compared to BSA powder. It shows an additional sharper contribution at 1.37 Å⁻¹ (4.58 Å) that leads to a strong asymmetric profile. This is compatible with an increase of the β-sheet content with intra or inter-molecular origin. In conclusion, the secondary structures of BSA are preserved in the prepared material with a slight modification of the α/β structures ratio. These findings support the results obtained by FTIR amid I band analysis.

SEM Surface Analysis

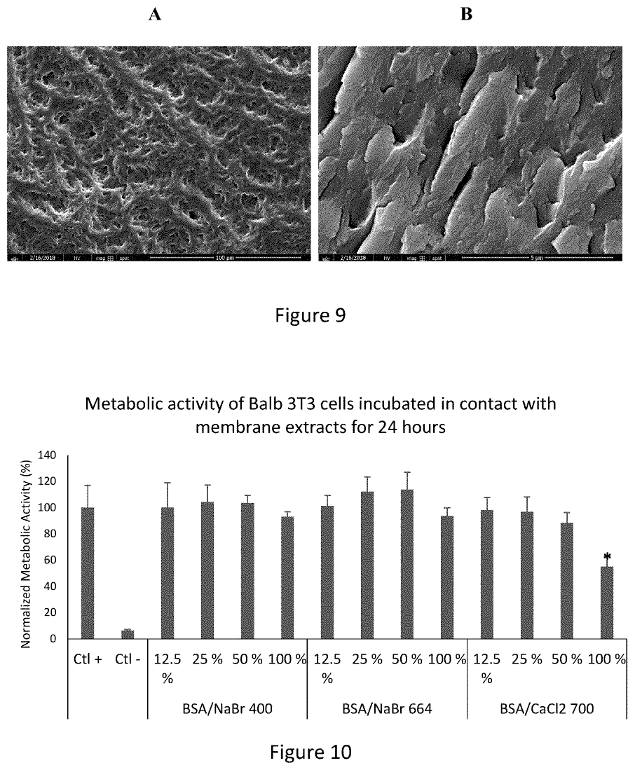

In order to study the surface topography of membranes formulated with NaBr (molar ratio NaBr/BSA=664) and to evaluate their porosity, the membranes were analyzed using SEM. The surface of the membrane seems to be highly rough with many pores and pore-like substructures (A, FIG. 9). An observation of a slice of this same membrane (cross-section) shows the existence of small pores inside the biomaterial. However, there does not seem as much as one might think by observing the surface (B, FIG. 9). The surface irregularities are mostly due to the evaporation used for formulation.

Other Albumin Proteins

The formulation procedure developed produced albumin-based membranes using BSA with a good repeatability. Formulations with other albumin proteins were carried out to study the feasibility of these membranes. Two albumins have been selected, human serum albumin (hSA), which has a very similar structure to bovine serum albumin, and ovalbumin (OVA), which has a structure and a molecular weight different from the two other proteins. Interesting membranes were obtained with both hSA and OVA. These have a different morphology than BSA membranes. In addition, they are more prone to hydration, resulting in higher water content and initial swelling. In conclusion, the established formulation process allows the preparation of albumin-based membranes regardless of the origin of the protein. However, only the BSA-based membranes were further characterized in this study due to the lower cost of this protein and its close resemblance to the human one.

Other membrane formulations were tested with different proteins, as γ-globulins. The main constraint was the protein solubility in water. In fact, albumin has an unmatched solubility in water allowing the preparation of solutions at very high concentrations. If the protein concentration should be lowered because of the protein's solubility threshold, the volume required to reach the minimal thickness compatible with membrane handling should be increased, prolonging considerably the evaporation processing. Membranes of γ-globulin (solubility in water at 20° C.≈20 mg/mL) were successfully formulated by evaporating a solution of bovine γ-globulin and NaBr (molar ratio salt/protein=664, M/S=35 mg/cm²). Therefore, the formulation procedure developed in this work proves to be promising for the formulation of protein-based membranes.

Biological Assays

Cytotoxicity and Cell Adherence

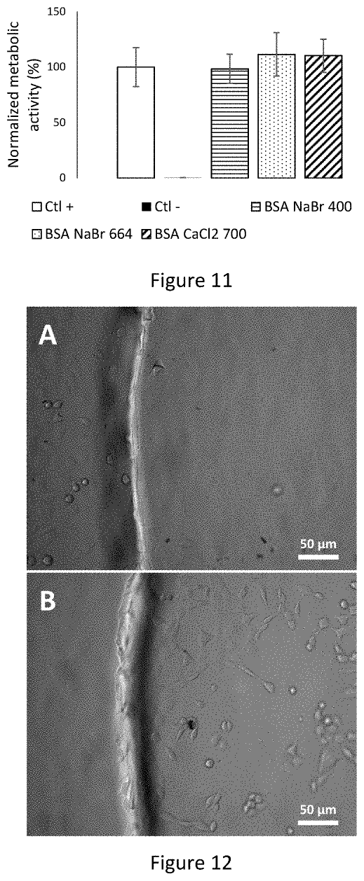

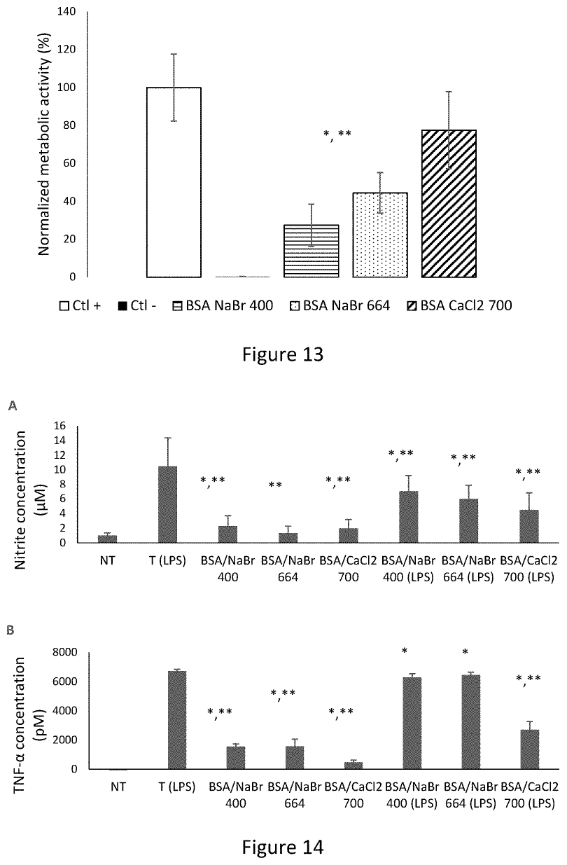

The biological properties of BSA/NaBr 664 membranes were studied. Two other interesting membranes were included in these experiments to have comparable references, BSA/NaBr 400 and BSA/CaCl₂) 700. First, the cytotoxicity of the membranes leachable components was evaluated by incubating Balbc 3T3 mouse fibroblasts in membrane extracts. For this cell line, the cell viability can be estimated by measuring the metabolic activity. The normalized metabolic activity of the cells cultivated with each extract was then compared to the metabolic activity of untreated cells (positive control). For this experiment, the extracts were diluted to reveal any dose-dependent effect. The statistical analysis of the obtained data concluded in no significant difference between the metabolic activity of the untreated group and the groups treated with various dilutions of the BSA/NaBr membrane extracts (see FIG. 10). Furthermore, the non-cytotoxicity of albumin and the absence of NaBr in the final membranes as shown previously match the observed non-cytotoxicity of the leachable component of the BSA/NaBr membranes. Then, the direct cytotoxicity was assessed by incubating Balbc 3T3 cells directly with these membranes. Relatively thin membranes (average thickness≈0.7 mm) were used in this experiment to allow the follow-up of the interaction between the cells and the material by microscopy (see FIG. 11). The statistical analysis of the data showed no significant difference between the metabolic activity of the untreated cells and the cells cultivated with the tested membranes (see FIG. 10). Furthermore, the microscopic examination revealed that the fibroblasts were spreading around as well as above the membranes (see FIG. 12). To quantify cellular adherence on the membranes, Balbc 3T3 cells were incubated for 24 hours with the membranes. Then, the culture media was eliminated, the membranes were transferred to an empty well and the metabolic activity of the cells on each membrane was measured. A significant difference was observed between the BSA/NaBr 664, the BSA/NaBr 400 and the BSA/CaCl₂ 700 groups (p<0.05). Around 45% (44.55%±10.67%) of the fibroblasts seeded adhered on the BSA/NaBr 664 membranes. The estimated percentage was lower for the BSA/NaBr 400 membranes (27.36%±11.22%) and higher for the BSA/CaCl₂ 700 membranes (77.62%±20.26%) (see FIG. 13). Therefore, the initial formulation solution seems to have a significant effect of the interaction of the cells with the membrane despite the complete elimination of the salt during the washing process. In conclusion, the formulated albumin-based biomaterials are non-cytotoxic and are in favour of cell adhesion and colonization.

Macrophage Activation

The effect of the albumin membranes on macrophage activation was evaluated by measuring nitrite and TNF-α concentration. NO and TNF-α are produced by activated macrophages to initiate and sustain the inflammatory response. Raw macrophages were cultivated with the tested membranes for 24 h. Afterwards, LPS was introduced directly in the wells of the LPS-activated groups to activate the macrophages and the cells were incubated for another 24 h. In comparison with the non-treated group (NT), nitrite production undergoes a slight increase in the presence of the tested membranes (p<0.05). The LPS activation induces a significant increase of the nitrite concentration in the culture media. However, in comparison with the LPS-treated control (T LPS), nitrite production seems to be significantly lower when the macrophages are cultivated with albumin membranes (p<0.05). TNF-α production follows a similar trend for the inactivated groups. Although, there is no significant difference between TNF-α production in BSA/NaBr groups and the T LPS group, unlike the BSA/CaCl₂ 700 group that showed a significantly lowered TNF-α production (p<0.05). Therefore, the tested albumin-based membranes do not efficiently induce the inflammatory response by activating macrophages.

### CONCLUSION

In these tests, several interesting biomaterial models have been developed with the following salts: NaBr, NaI, KI, CaCl₂), MgCl₂, potassium acetate and ammonium formate. The properties of these membranes could be modulated by modifying the formulation parameters. Two parameters of importance were identified: the presence of salt and the salt/albumin molar ratio. Also, it is possible to obtain membranes with ternary systems salt1/salt2/albumin or salt/albumin/polymer, which were successfully formulated. These systems make it possible to modulate the properties of the membranes (mechanical and intrinsic properties) and to obtain functionalized biomaterials with new properties. Albumin-based biomaterials formulated by evaporation in the presence of salt form a versatile model whose properties can be modulated to adapt to the requirements of targeted therapeutic applications.

### Example 2: Physicochemical Investigation and Assessment of the Versatility of the Technology, Loading of Active Substances, and Preliminary Evaluation of the Biocompatibility and of the Biodegradability In Vivo

2.1 Physicochemical Investigation and Assessment of the Versatility of the Technology

For the following experiments, BSA/NaBr materials were prepared by evaporation at 37° C. of a solution of BSA (initial concentration=100 mg/mL) and NaBr (initial concentration=62 mg/mL) prepared in a sodium acetate buffer (pH=6) until the formation of a dry material. As for BSA/CaCl₂) materials, they were prepared by evaporation at 37° C. of a solution of BSA (initial concentration=100 mg/mL) and CaCl₂ (initial concentration=155 mg/mL) prepared in a sodium acetate buffer (pH=6) until the formation of a dry material.

Stability in organic solvents. The majority of active substances being lipophilic molecules, organic solvents are very useful for their solubilisation and loading in biomaterials. Furthermore, drug loading in a readily formed material (post-loading) requires the stability of the material in the solvent used to solubilise the drug. The stability of Albupad materials (i.e. the biomaterial according to the invention) in organic solvent was assessed in the following solvents: ethanol, DMSO, acetonitrile and dichloromethane. BSA/NaBr and BSA/CaCl₂ membranes were placed in each solvent for 72 h at room temperature. Their stability was assessed by comparing their mass loss. The membranes presented no mass loss after incubation for 72 h in all the tested solvents. Furthermore, their physical aspect did not show any visible signs of degradation and their hydration properties were also preserved. Thus, Albupad materials are stable in ethanol, DMSO, acetonitrile and dichloromethane.

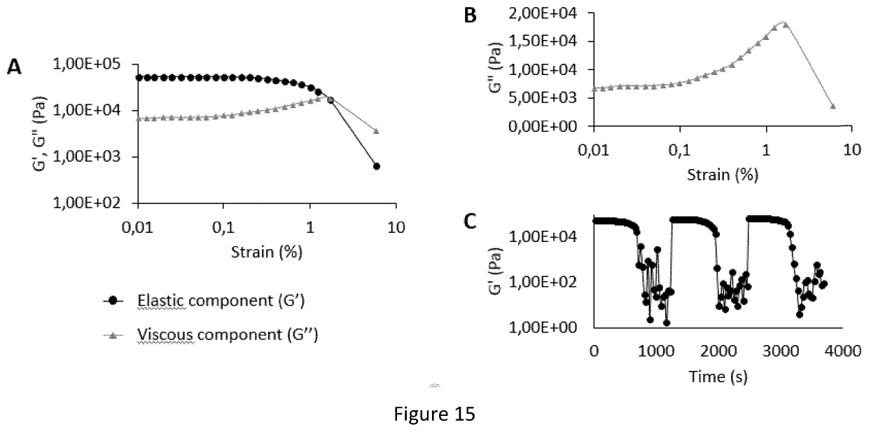

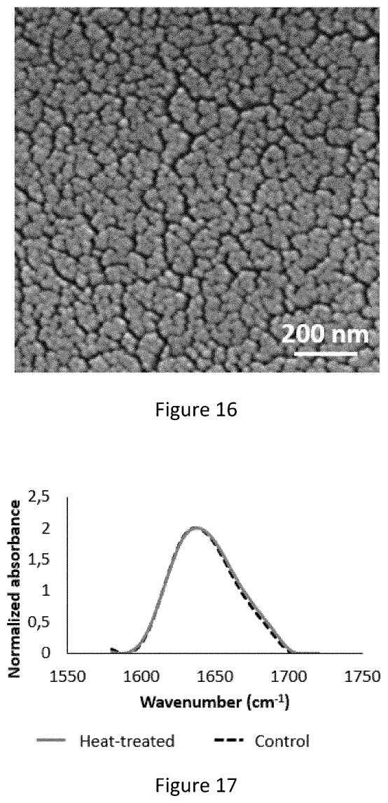

Rheological evaluation of BSA/NaBr membranes. In this study, amplitude sweep tests were performed on hydrated (in water) BSA/NaBr membranes (n=4) to study their viscoelastic behavior. G′ represents the elastic or recoverable component, and G″ is the viscous component. During each test, the frequency was set at 0.5 Hz while the strain was increased from 0.01 to 100%. A strong sliding effect was observed beyond a strain of 10% without any noticeable damage in any of the tested samples, causing the data to be unusable (omitted). In the linear viscoelastic region (LVE) under 1% strain, G′ (59.6±5.1 kPa) is higher than G″ (7.7±0.8 kPa) (FIG. 15A). Therefore, the material behaves as a solid elastic material in this range. After 1% strain, a Payne effect is identified as G″ increases and reaches a maximum at a strain of 1.79±0.07% (FIG. 15B). This effect is characteristic of materials made up of two phases, a matrix in which harder particles are suspended causing greater energy dissipation at certain deformations. This effect has been described primarily in rubber elastomers loaded with carbon black particles and is attributed to the changes induced by the deformation of the microstructure of the material. Thus, BSA/NaBr materials consist of a matrix containing harder particles. However, it was determined that the salt was eliminated during the washing process. Therefore, the particles are likely albumin aggregates/particles surrounded in a softer albumin matrix. This result was confirmed by MEB analysis of the section of a BSA/NaBr material, revealing a particulate structure forming these materials (FIG. 16). When the material is subjected to multiple consecutive amplitude sweep tests (FIG. 15C), G′ recovers its initial value, proving that the cohesion of the material was not altered during the experiment. Similar observations were made with several other formulations such BSA/CaCl₂) membranes, showing that these rheological findings are a property characteristic of Albupad materials.

Contact angle of BSA/NaBr membranes. The contact angle measurement was performed by depositing a micro drop of milliQ water (5 μL) on the surface of a dry sample of BSA/NaBr membrane (deposition rate=2 μl/s). Images were recorded for 100 s (1.4 FPS). During these tests, contact angle varying between 90° and 100° were measured. The kinetics of hydration of the membrane and of sinking of the drop into the material was slow (>30 min) and therefore could not be observed during these measurements. Therefore, the surface of a dry BSA/NaBr membrane is moderately hydrophilic due to slow hydration kinetics and high surface roughness (observed by SEM analysis in previous work).

Accelerated aging of BSA/NaBr membranes. A dry BSA/NaBr membrane was placed in an oven at 80° C. for 3 days. Amid I spectra of the heat-treated membrane was then analyzed using FT-IR and compared to a control membrane. Amid I band of the heat-treated membrane was similar to that of the non-treated control (FIG. 17). Therefore, the secondary structure of albumin in the membrane was not altered by heating in the tested conditions.

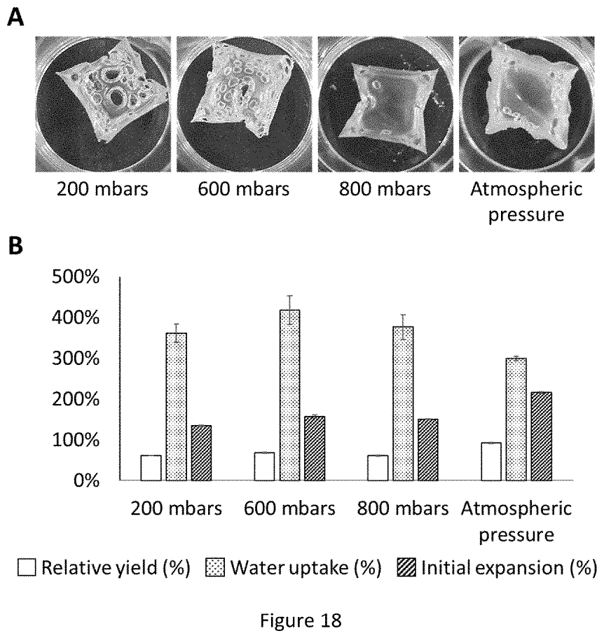

Production under controlled vacuum. The production of Albupad material by evaporation under vacuum can provide a useful tool to reduce the time required for the evaporation. Therefore, the feasibility of the formulation under controlled pressure was performed using a vacuum oven. Solutions of BSA/NaBr (initial concentration of BSA=100 mg/mL, initial concentration of NaBr=1 M) were evaporated at 37° C. in a vacuum oven for 24 h and the following pressure values were tested: 800, 600 and 200 mbars. The dry materials were then washed and soaked in water for 48 h in order to assess their stability. Furthermore, physical appearance, relative yield, water uptake and initial expansion values were used to characterize the materials formed under controlled vacuum and compare them to a control batch prepared under atmospheric pressure. Handable and water insoluble materials were obtained after evaporation of BSA/NaBr solutions under controlled vacuum at all tested pressure values. The formed membranes had a similar physical aspect and handability to the control batch. However, soluble gas elimination from the solution during membrane formation let to the entrapment of bubbles in the membrane structure leading to the formation of large pores at 200 and 600 mbars (FIG. 18A). Therefore, the optimization of the formulation is required in order to control or prevent the formation of these pores, by adding a step of solution degassing before the evaporation for example. Furthermore, the relative yields of the formulation under vacuum (60% to 70%) are relatively lower than the control batch (>90%) (FIG. 18B). Next, the mechanical and rheological properties of the membranes prepared at 600 mbars were assessed. A compression assay was performed on hydrated BSA/NaBr 600 mbars hydrated (in water) membranes using a rheometer in parallel-plate measuring system. The elastic modulus of the membranes was 0.76 MPa, similar to the elastic modulus of the control batch and to the elastic modulus previously measured for the BSA/NaBr material. Amplitude sweep tests were performed on hydrated (in water) BSA/NaBr membranes (n=4) to study their rheological properties. During each test, the frequency was set at 0.5 Hz while the strain was increased from 0.01 to 100%. These experiments revealed that the BSA/NaBr membranes formulated under controlled vacuum at 600 mbars had similar rheological properties as BSA/NaBr membranes formulated under atmospheric pressure. Therefore, the formulation under controlled vacuum allows the formation of Albupad materials without altering their properties.

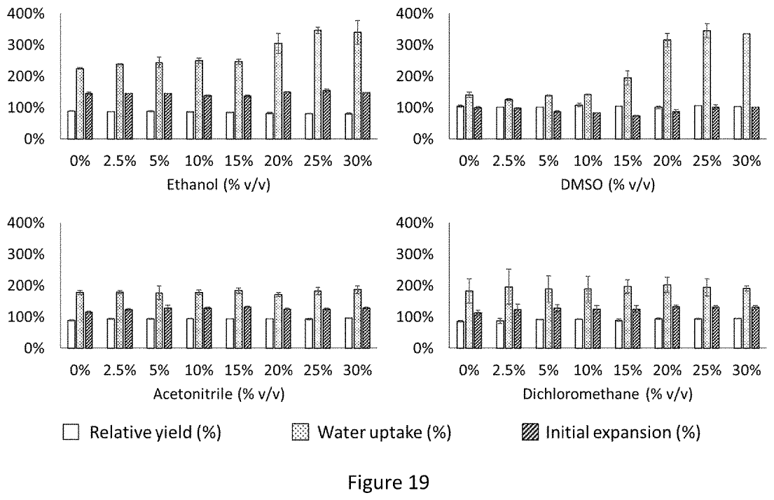

Solvent investigation. The use of an organic solvent such as ethanol, DMSO or acetonitrile could provide an interesting tool to incorporate active substances with poor water-solubility into the solution of albumin and salt, thus providing a way to pre-load these molecules in Albupad materials. The organic solvent will be readily eliminated during the evaporation step (for volatile solvents such as ethanol), or during the washing step (for water miscible solvents such as DMSO). The study of the feasibility of the formulation of Albupad materials in a mixture organic solvent/albumin solution was therefore tested. For this investigation, four solvents were selected, ethanol, DMSO, acetonitrile and dichloromethane. The solvents were added directly in BSA/NaBr solutions according to the following volume ratios solvent/solution: 2.5, 5, 10, 15, 20, 25 and 30% v/v. The solvent/solution mixtures are then evaporated at 37° C. for 7 days. Subsequently, the dry materials were washed in distilled water for 48 hours. The relative yield, water uptake and initial expansion of the formulated materials were compared to control batches formulated without organic solvents (volume ratio=0%). Stable and water insoluble membranes were formed in the presence of the four solvents at all tested ratios. These materials were handable and shared a similar physical aspect to the control batches. For the materials formulated in the presence of ethanol, the membranes prepared with volume ratios ranging from 2.5% to 15% have similar relative yields (85-88%), water uptake (238-250%) and initial expansion (137-145%) values to the control batch (FIG. 19). The membranes prepared with volume ratios ranging from 20% to 30% show a decrease of relative yields (80-82%) and an increase of water uptake (304-346%) and initial expansion values (147-154%) when compared to the control batch (FIG. 19). The presence of DMSO did not have an influence on the relative yield of the formulation or the initial expansion values of the materials. The water uptake values of the membranes prepared with volume ratios ranging from 2.5% to 10% were comparable to the control batch, meanwhile those of the membranes prepared with volume ratios ranging from 15% to 30% were higher (194-345%) (FIG. 19). As for the tested non-water miscible solvents (acetonitrile, dichloromethane), all the prepared membranes shared similar relative yields, water uptake and initial expansion values (FIG. 19). In conclusion, the presence of up to 30% (v/v) of an organic solvent does not prevent membrane formation and allows the preparation of handable materials. No modification of the properties of these materials was observed in comparison to the control batch for up to 15% ethanol, 10% DMSO and 30% acetonitrile or dichloromethane. Similar results were obtained in the case of BSA/CaCl₂) membranes as well as BSA/NaBr and BSA/CaCl₂) sponges.

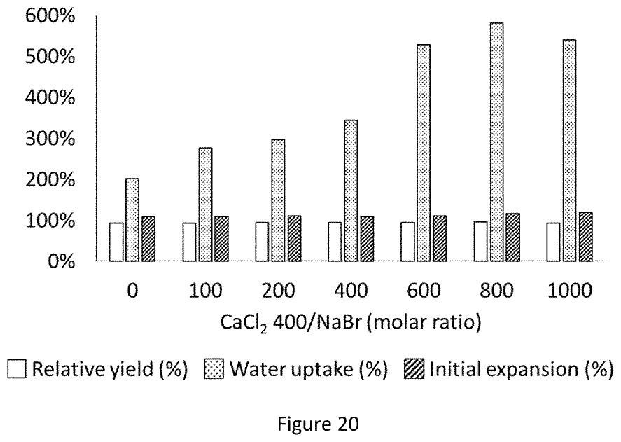

Combination of salts. Salt being a key parameter for the formulation of albumin materials using Albupad technology, the type of salt and its concentration were relevant tools to modulate the materials properties. Furthermore, salt combinations could provide additional tools for better tuning of the materials properties, allowing the Albupad materials platform to be more flexible and adaptable according to the requirements of a potential application. Therefore, the applicability of Albupad technology was investigated for the formulation of albumin materials using a combination of salts. For these experiments, various combinations of two different salts were tested: a primary salt (S1) and a secondary salt (S2). The molar ratio salt/albumin of S1 was set while the molar ratio salt/albumin of S2 was varied from 100 to 1000. The following combinations of salts were tested: NaBr 400/CaCl₂), CaCl₂ 400/NaBr, NaBr 400/MgCl₂, NaBr 50/NaCl, NaBr 400/NaCl, CaCl₂ 400/MgCl₂ and NaCl 400/KCl. Handable membranes were obtained with the combinations NaBr 400/CaCl₂), CaCl₂ 400/NaBr, NaBr 400/MgCl₂, NaBr 400/NaCl, CaCl₂ 400/MgCl₂ at all tested S2 molar ratios. Among the tested salts, the salts NaBr, CaCl₂ and MgCl₂ allow the formation of handable and stale albumin materials. As a matter of fact, albumin membranes were obtained if the combination of salts includes at least one salt that allows membrane formation and if the molar ratio salt/albumin of this salt is sufficient (>100). Relative yield, water uptake and initial expansion were used to characterize the formed material in order to evaluate the effect of the secondary salt on the properties of the material. FIG. 20 represents the membranes formulated with the combination CaCl₂ 400/NaBr. NaBr molar ratios in the range 100-1000 were tested and a control prepared only with CaCl₂ and BSA at a molar ratio salt/albumin of 400 was prepared. The addition of NaBr into the formulation did not modify the relative yield of the formulation. However, it generated a notable increase of the water uptake. The modification of the membrane properties was also noted with the other combinations. Furthermore, the feasibility of the formulation with a combination of three salts was also tested. These experiments showed that stable and handable membranes could be produced with NaBr/CaCl₂/MgCl₂ combinations (tested molar ratios: 100/100/100, 200/200/200, 300/300/300). In conclusion, the use of multiple salts does not prevent membrane formation and provides a relevant tool for fine adjustment of Albupad materials properties.

Protein investigation. Four batches of human serum albumin (HSA) were tested: HSA FAF (fatty acid free), HSA LFP (low folate powder), HSA RGP (reagent grade powder) and rHSA (recombinant HSA, from rice). In addition, two globular proteins, γ-globulin (human origin) and hemoglobin (human origin) were tested. The solubility of the protein being the major criteria of applicability of Albupad technology, these globular proteins were selected because of their adequate water solubility. Each protein was tested with the following salts: NaBr, NaCl and CaCl₂ (molar ratios salt/protein: 0-2000). Solutions of protein and salt were evaporated in an oven at 37° C. for 7 days. The dry material was then washed and soaked in water for 48 hours to eliminate the salt and select the water-insoluble material. Water-insoluble membranes were obtained with all human albumin proteins with NaBr and CaCl₂. These membranes were stable and handable. γ-globulin-based membranes were properly formed with NaBr. Thus, Albupad technology can be used to produce stable and handable materials with human serum albumin and recombinant human albumin, as well as human γ-globulin.

2.2 Loading of Active Substances

Due to the inherent capacity of albumin to load various substances, albumin based materials are promising for drug delivery and controlled release. Furthermore, Albupad technology allows the loading of active substances before the formation of the material in the initial solution (pre-loading), as well as the loading of active substances in the material after its formation (post-loading). A preliminary investigation revealed that Albupad materials could be pre-loaded with lipophilic substances such as piroxicam and fluticasone, as well as chlorhexidine, a water-soluble molecule. In this investigation, stable and handable materials were formed, leading to the validation of the applicability of this loading strategy to the Albupad technology. Furthermore, doxorubicin (DOX), an antitumor drug, as well as insulin (INS), a peptide used in the treatment of diabetes, were also loaded in Albupad materials in order to establish a proof of concept and study the release of this substances overtime. The loading of these substances was assessed by fluorescence imaging. Drug release quantification in water was performed by fluorescence titration overtime.

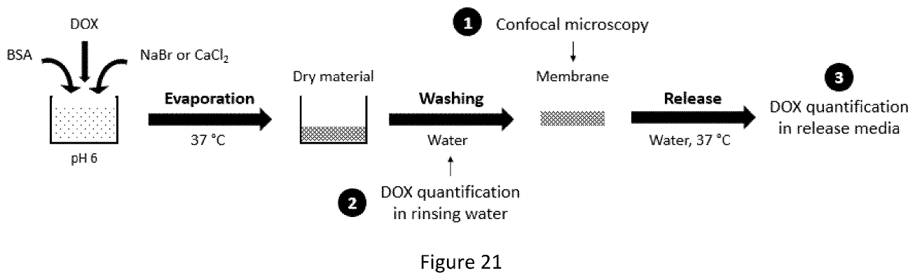

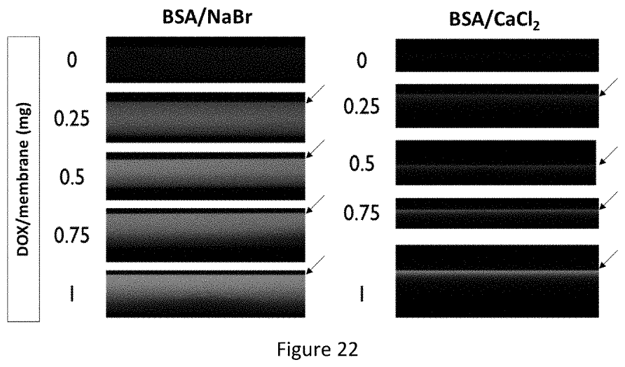

Loading and release of DOX pre-loaded in Albupad membranes. BSA/NaBr and BSA/CaCl₂ membranes were pre-loaded with DOX by incorporating the latter directly in the solutions before evaporation as in FIG. 21. Different quantities of DOX were incorporated in the solutions as follows: 0.25, 0.5, 0.75 and 1 mg of DOX per membrane (membrane mass≈400 mg). Handable membranes were obtained after evaporation of the solutions. The prepared membranes appeared colored due to the presence of DOX. Moreover, the intensity of the colouration of the membranes was proportional to the quantity of the loaded DOX loaded. In order to visualise the presence of DOX inside the membranes, CLSM imaging was performed (FIG. 22). In the BSA/NaBr membranes, the fluorescence of the DOX was evenly distributed inside the matrices of the membranes (thickness=500 μm thickness), showing that the active substance was successfully loaded in the material (FIG. 22). As for the BSA/CaCl₂ membranes, the opacity of the materials prevented the observation of the DOX loaded inside the materials matrices. However, DOX fluorescence was observed on their surfaces, revealing that the loading of DOX in these membranes was also successful (FIG. 22).

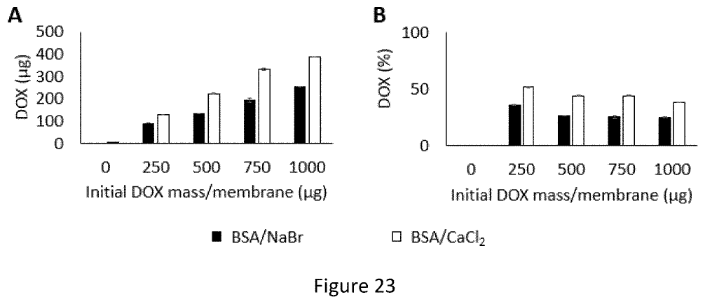

Next, the membranes were washed in water in order to eliminate the salt. BSA/NaBr membranes were washed in water (3×20 mL) for 2 hours. BSA/CaCl₂ membranes were washed in water (4×20 mL) for 2 hours. The rinsing solutions were collected and the quantity of the eliminated DOX was measured by fluorescence spectroscopy at 485 nm (FIG. 23). The eliminated DOX was between 20% and 30% for the BSA/NaBr membranes and between 35% to 50% for the BSA/CaCl₂ membranes. Hence, 50% to 70% of the DOX initially loaded was still available in the membranes after washing.

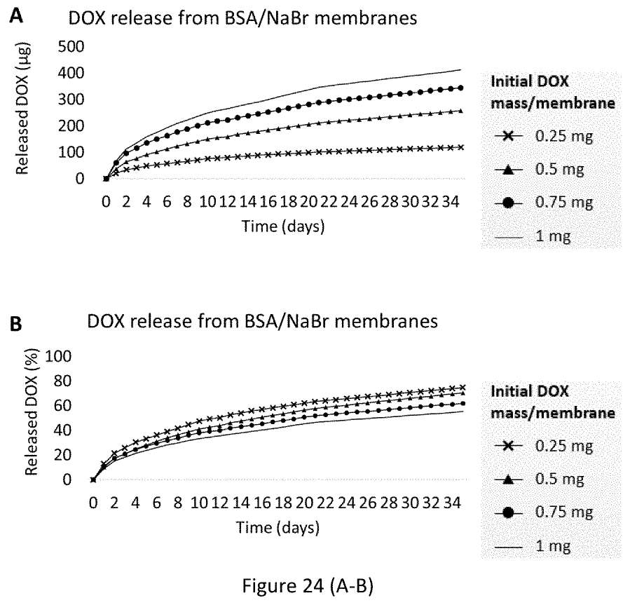

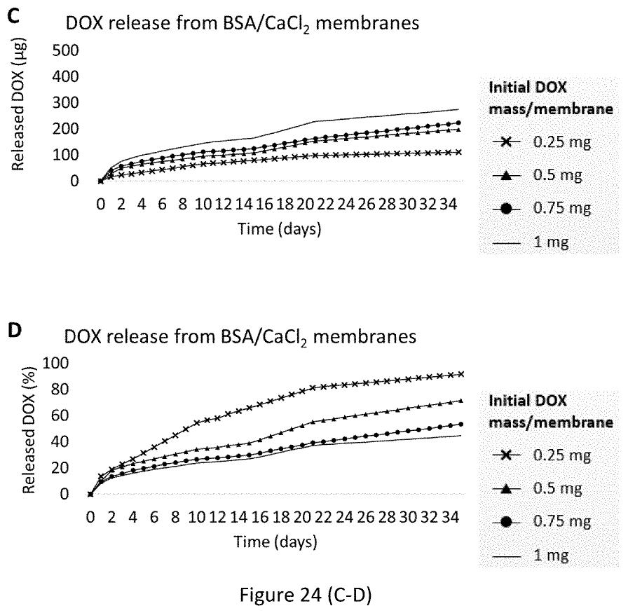

DOX release from the membranes was investigated in water at 37° C. for 35 days under stirring. DOX titration in the supernatant over 35 days revealed that the release profile of this active substance from Albupad materials was slow and controlled with a limited burst effect (FIG. 24). Moreover, 10 to 50% of the available DOX left unreleased after 35 days depending on the formulation, showing the potential of the material for drug delivery.

Loading and Release of INS-FITC Pre-Loaded in Albupad Membranes.

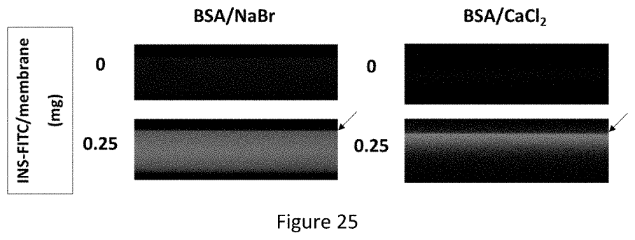

BSA/NaBr and BSA/CaCl₂ membranes were pre-loaded with INS-FITC by incorporating the latter directly in the solutions before evaporation. The quantity of INS-FITC were incorporated in the solutions was 0.25 mg per membrane (membrane mass≈400 mg). Handable membranes were obtained after evaporation of the solutions. In order to visualise the presence of INS-FITC inside the membranes, CLSM imaging was performed. In the BSA/NaBr membranes, the fluorescence of the INS-FITC was evenly distributed inside the matrices of the membranes (thickness=500 μm thickness), showing that the active substance was successfully loaded in the material (FIG. 25). As for the BSA/CaCl₂ membranes, the opacity of the materials prevented the observation of the INS-FITC loaded inside the materials matrices. However, fluorescence was observed on their surfaces, revealing that the loading of DOX in these membranes was also successful (FIG. 25).

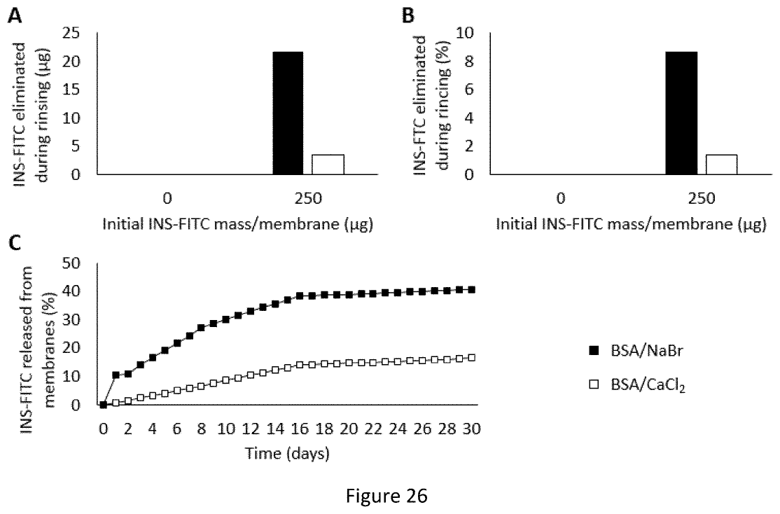

Next, the membranes were washed in water in order to eliminate the salt. BSA/NaBr membranes were washed in water (3×20 mL) for 2 hours. BSA/CaCl₂ membranes were washed in water (4×20 mL) for 2 hours. The rinsing solutions were collected and the quantity of the eliminated INS-FITC was measured by fluorescence spectroscopy at 495 nm (FIGS. 2.7A and B). For BSA/NaBr and BSA/CaCl₂ membranes, respectively 8% and 1% of the loaded INS-FITC were eliminated during washing process. Hence, more than 90% of INS-FITC was available in the membranes after the washing process. Subsequently, INS-FITC release from the membranes was investigated in water at 37° C. for 30 days under stirring. In accordance to the results obtained with DOX, INS-FITC titration in the supernatant over 30 days revealed that the release profile of this active substance from Albupad materials was slow and controlled with a limited burst effect (FIG. 26C). Moreover, 60 to 80% of the available INS-FITC left unreleased after 30 days depending on the formulation, showing the potential of the material for the extended release of this active substance.

In conclusion, considering all the performed experiments, the feasibility of loading material using pre-loading method was proven. Furthermore, other active substances were also successfully loaded in Albupad membranes such as gentamycin with a loading rate as high as 30% (wt/wt). Moreover, the maximal loading rate is dependent on the solubility of the active substance and its interaction with albumin. Hence, further investigations should allow the study and optimisation of the loading of targeted active substances. Furthermore, the presence of protease in the release medium should allow the increase of substance release from the membranes.

2.3 Preliminary Evaluation of the Biocompatibility and the Biodegradability In Vivo

Optimisation of the sterilisation of Albupad materials using electron beam irradiation. Prior the in vivo implantation of Albupad materials, the sterilisation using electron beam irradiation was tested in order to verify its compatibility with the material, thus not causing its degradation or significant change in its properties or of the structure of albumin composing it. Therefore, two types of membranes were prepared: BSA/NaBr and BSA/CaCl₂. The irradiation of these samples was divided in groups in order to test their stability under irradiation in the following conditions: in the presence or not of a radioprotective agent (vitamin C) and in the form of dry or hydrated (in water) membranes. Four irradiation doses were tested: 5, 10, 25 and 25 kGy. The physical aspect, mass loss and water uptake of the irradiated membranes were compared to a non-irradiated control batch. Furthermore, the IR spectra of the irradiated membranes were compared to those of the control in order to detect alteration of albumin's secondary structures. These experiments revealed that the irradiation of the materials did not cause their degradation at all tested irradiation doses, and that the use of vitamin C as a radioprotective agent was not necessary. The comparison between the irradiated samples and the control batch showed no significant modification of the properties of the materials after their irradiation. Additionally, FT-IR analysis showed that the irradiation of the materials did not modify amid I band, therefore not damaging albumin's secondary structures, in the dry samples, as well as in the hydrated samples. In conclusion, the sterilisation by electron beam irradiation is compatible with Albupad materials and could be efficiently used at 25 kGy to sterilise these materials prior to their in vivo evaluation.

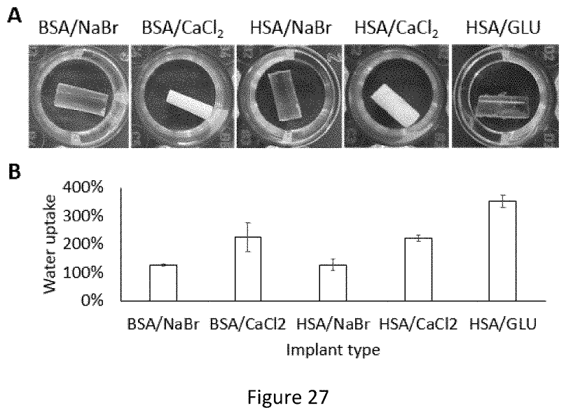

Implant formulation, sterilisation and subcutaneous implantation in mice. In order to carry on a preliminary investigation of the in vivo biodegradability and the biocompatibility of Albupad materials in vivo, the following batches of cylindrical implants were prepared: BSA/NaBr (initial concentration of BSA=300 mg/mL, initial concentration of NaBr=1.8 M, n=5), BSA/CaCl₂ (initial concentration of BSA=200 mg/mL, initial concentration of CaCl₂=2.1 M, n=10), HSA/NaBr (initial concentration of HSA=300 mg/mL, initial concentration of NaBr=1.8 M, n=5) and HSA/CaCl₂ (initial concentration of HSA=200 mg/mL, initial concentration of CaCl₂=2.1 M, n=10). A control batch of implants was prepared by crosslinking HSA in the presence of glutaraldehyde (HSA/GLU, n=5). The implants were then thoroughly washed and cut to fit the size recommendation (length≈1 cm, diameter <5 mm) (FIG. 27A). The water uptake of these materials was used to compare the batches prepared with BSA and HSA. BSA/NaBr and HSA/NaBr shared similar visual aspect and water uptake values (124±3% and 127±21% respectively), as well as BSA/CaCl₂ and HSA/CaCl₂ (224±52% and 220±10% respectively) (FIG. 27B). The implants were then dried in 24-well plates at 37° C. for 24 h. The dry batches of implants were then sterilised using the selected method (electron beam, 25 kGy).

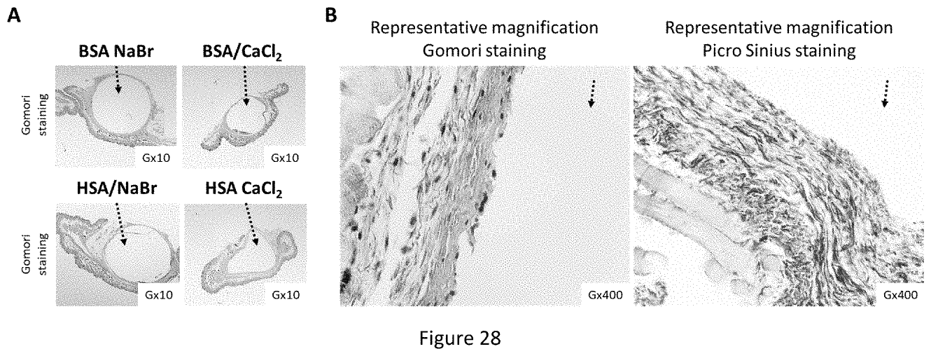

Next, the sterilised implants were used for subcutaneous implantation in mice. The implants were rehydrated in PBS at 4° C. for 6 hours, then rinsed with PBS before implantation. Swiss mice were implanted with BSA/CaCl₂, HSA/CaCl₂ and HSA/GLU implants (three mice per type of implant). Nude mice were implanted with BSA/NaBr, BSA/CaCl₂, HSA/NaBr and HSA/CaCl₂ implants. Acute toxicity was observed in the control group implanted with BSA/GLU materials and led to the sacrifice of the mice of this group. The mice implanted with Albupad materials stayed alive throughout the experiment (28 days) with no significant weight variation (±5-10% weight variation). The volume of the implants was measured through their skin using a caliper. In Swiss mice groups, BSA/CaCl₂ implants were completely degraded after 17 days of implantation; meanwhile HSA/CaCl₂ implants were not entirely degraded by the end of the experiment after 28 days and lost in average 50% of their volume. In Nude mice, BSA/NaBr, HSA/NaBr, BSA/CaCl₂ and HSA/CaCl₂ implants lost respectively 23%, 35%, 53% and 43% of their volume after 28 days of implantation. After sacrifice of the mice at day 28, histological evaluation of cuts of the implants and their surrounding tissue was performed by microscopy after Gomori and Picro Sinius staining. The observation of the prepared cuts revealed that the implants were not fragmented, showing that their biodegradation is progressive and does not lead to the formation of large fragments (FIG. 28A). It was also observed that the phenotype of the tissue, surrounding the implants, was normal and that the formation and orientation of collagen fibers were similar to those observed in native tissue (FIG. 28B). No fibro-proliferation was observed in contact with the material. Therefore, the tested implants were nontoxic for the surrounding tissues or the mice and proved to be biodegradable in vivo with rates depending on the type of implant.

Experimental Section

Chemical reagents. Bovine serum albumin (fraction V, >96%) was purchased from Acros Organics. From Sigma-Aldrich were purchased hemoglobin (human), γ-globulins from bovine blood (99%), recombinant human albumin (rHSA, expressed in rice), sodium bromide (NaBr), potassium chloride (KCl), Doxorubicin (DOX), insulin conjugated to FITC (INS-FITC) and deuterium oxide (D₂O). Sodium chloride (NaCl) was purchased from VWR Chemicals. Magnesium chloride (MgCl₂, anhydrous) was purchased from Fluka. Calcium chloride (CaCl₂, 2H₂O) was purchased from Merck. From Sera Care were purchased the following human serum albumins (HSA): HSA reagent grade powder (RGP), HSA low folate powder (LFP) and HSA fatty acid free (FAF).

Formulation (general procedure). A solution of BSA (100 mg/mL) and NaBr (1 M) in a sodium acetate buffer (0.2 M) at pH 6 was prepared. This solution was deposited in a silicone mold and evaporated at 37° C. until dry. The dry biomaterial obtained was washed to remove the salt and soaked in distilled water at room temperature for 48 hours. The water-insoluble membrane (BSA/NaBr) was then collected and characterised.

Initial characterisation. The relative yield (Equation S1), the water uptake (Equation S2) and the initial expansion (Equation S3) were used to compare the formulated membranes. W_{BSA} represents the initial mass of albumin used for the formulation. Aᵢ represents the area of the circular container used during the evaporation process. W_{d} represents the mass of the final dried membrane after washing in distilled water for 48 hours and drying in an oven at 37° C. overnight. V_{d} is the volume of the dried membrane measured by immersion of the material in distilled water at room temperature. Wₕ represents the mass of the hydrated membrane at equilibrium after immersion in distilled water for 24 h and removal of excess surface water using filter paper. Aₕ is the area of the hydrated membrane's surface at equilibrium after immersion in distilled water for 24 h, calculated after measuring its diameter using an electronic digital caliper (TACKLIFE-DC01, accuracy±0.2 mm).

$$Relative yield ( Y \% ) = \frac{{W}_{d}}{{W}_{BSA}} ⨯ 100 \qquad ( S1 )$$

$$Water uptake ( W \% ) = \frac{{W}_{h} - {W}_{d}}{{W}_{d}} ⨯ 100 \qquad ( S2 )$$

$$Initial expansion ( E \% ) = \frac{{A}_{h}}{{A}_{i}} ⨯ 100 \qquad ( S3 )$$

Scanning Electron Microscopy (SEM). Scanning electron microscopy and microanalysis assessments were performed using a Quanta™ 250 FEG SEM (FEI Company, Eindhoven, The Netherlands) operating with an accelerated voltage of electrons of 10 kV. For SEM experiments, BSA/NaBr 664 membranes were dried then coated with a gold-palladium alloy using a Hummer Jr sputtering device (Technics, Union City, Calif., USA).

Stability in organic solvents. BSA/NaBr membranes were placed in 5 mL of the following organic solvents: DMSO, acetonitrile and dichloromethane. The media containing the membranes were then incubated at room temperature under stirring for 72 h. After that, the membranes were washed with water before being characterised for their mass loss (Equation S4).

$$Mass loss ( ML \% ) = \frac{Initial mass ( dry ) - Final mass ( dry )}{Initial mass ( dry )} ⨯ 100 \qquad ( S4 )$$

Mechanical properties of the membranes. The compression testing was performed using a rheometer Kinexus ultra+(Malvern, United Kingdom) in parallel plate configuration (20 mm diameter). A batch of three hydrated (in distilled water) BSA/NaBr membranes (thickness=0.7 mm) was used. The elastic modulus (E) was calculated within the elastic domain of the strain (ε)-stress (σ) curve (Equation S5).

σ=E×ε (S5)

Rheological measurements. The viscoelastic behaviour of BSA/NaBr membranes (thickness=0.7 mm) was assessed using a rheometer Kinexus ultra+(Malvern, United Kingdom) in parallel plate configuration (20 mm diameter). Membranes were previously hydrated in distilled water for 24 hours. The BSA/NaBr discs were loaded between the plates, and the gap was closed until the sample was in good contact with both plates (normal force <1 N). At the beginning of the experiment as well as between experiments, the samples were equilibrated for 5 minutes. During this time, the normal force decreased to values below 0.1 N in all samples. Amplitude sweep tests were conducted at the shear strains of 0.01-100% at a fixed frequency of 0.5 Hz at 25° C. The shear modulus G*(ω)=σ(ω)/γ(ω) follows from the ratio between stress (σ) and strain amplitude (γ). G*(ω)=G′+iG″ is a complex quantity with an elastic storage modulus (G′) and viscous loss modulus (G″). The absolute magnitude of the shear modulus, |G*|, was calculated using |G*|=(G′²+G″²)^{0.5}.

Contact angle measurements. Attension® Theta tensiometer along with OneAttension software from Biolin Scientific (Sweden) was used. The experiments were carried out in “sessile drop” mode for measuring static contact angle of water on dry BSA/NaBr membranes. A Hamilton microsyringe (needle diameter=0.7 mm) was used for dispensing the water droplet. Each measurement was done with a droplet of 5 μL of milliQ water (drop rate=2 μl/s) and repeated three times for each sample. A built-in camera captured a sequence of images of the sessile drop (frame rate=14 FPS), while measuring the tangent angle made by the drop at its intersection with the material. The mean contact angle (average of left and right contact angle) was recorded after 10 s, a time sufficient for the drop to stabilise.

IR analysis. FTIR experiments were performed on a Vertex 70 spectrometer (Bruker, Germany) using a DTGS detector. Spectra were recorded in the Attenuated Total Reflection (ATR) mode using single reflection diamond ATR by averaging 128 interferograms between 800 and 4000 cm⁻¹ at 2 cm⁻¹ resolution, using Blackman-Harris three-term apodisation and Bruker OPUS/IR software (version 7.5). Readily prepared BSA/NaBr membranes (dry) were finely ground and there is spectra were recorded. To decompose the amide I band region (1700-1600 cm⁻¹), the data processing was performed using OPUS 7.5 software (Bruker Optik GmbH). Prior to the curve fitting, the spectra were baseline-corrected on the amide I band region and then normalised using a normalisation “min-max” method. The number of subbands and their positions were determined from the fourth derivative of the spectra. Deconvolution was carried out according to the least-square iterative curve fitting program (Levenberg-Marquardt) using a Gaussian line-shape. For the final fits, in order to reduce the residual RMS error as much as possible (less than 0.005), heights, widths and positions of all bands were adjusted while at least one of these parameters was not allowed to change each time. Finally, second derivatives of the original and the fitted curve were compared to ensure the accuracy of curve fitting. The fractional areas of the fitted components were used to calculate the percentages of different secondary structure elements (α-helix, β-sheets, β-turns, and random coils) after their identification according to the literature.

Pre-loading formulation. BSA solution (100 mg/mL) was prepared in an acetate buffer (0.2 M) at pH 6 and blended with NaBr (NaBr/BSA molar ratio=400) or CaCl₂) (CaCl₂)/BSA molar ratio=700) salts. For the pre-loading of doxorubicin (DOX), the following quantities of DOX were added to the initial solution before evaporation: 0.25, 0.5, 0.75 and 1 mg per membrane (membrane mass=400 mg). For the pre-loading of insulin (INS-FITC), 0.25 mg of INS-FITC per membrane (membrane mass=400 mg) was added to the initial solution before evaporation. The solutions were deposited in an anti-adhesive silicone mold and evaporated at 37° C. for 48 hours. The dry biomaterials obtained were thoroughly washed in 3×20 mL of water for BSA/NaBr membranes and in 4×20 mL of water for BSA/CaCl₂) membranes in order to eliminate the salts.

Confocal laser scanning microscope characterisation (CLSM). In order to visualize the presence of DOX or INS-FITC inside the pre-loaded membranes, CLSM imaging was performed using a ZEISS LSM 710 confocal microscope. The excitation/emission wavelength for membrane imaging was fixed to 450/515 nm for both DOX and INS-FITC pre-loaded materials.

Release experiment. The release of DOX or INS-FITC pre-loaded membranes was monitored with a Genius XC spectrofluorimeter (SAFAS, Monaco). The release experiment was carried out at 37° C. in H₂O (10 mL per membrane). Fresh 10 mL water were added after each supernatant collection. The supernatants were analysed with the spectrofluorimeter. The wavelengths of excitation and emission for DOX and INS-FITC were λₑₓ/λₑₘ=485 nm/595 nm and λₑₓ/λₑₘ=495 nm/520 nm, respectively.

Statistical analysis. Data were analysed by using R (version 3.6.1, R Foundation for Statistical Computing, Vienna, Austria). The normality of distribution was determined with the Shapiro-Wilk test. The equality of variances was determined with the F-test. When the data were normally distributed and the variances of the samples were equal, a t-test (2-tailed) was used to compare two means. When these two conditions did not apply, a Mann-Whitney test was performed instead. Values were considered statistically significant at p<0.05.

1. A process for preparation of a biomaterial comprising the steps of:
a) preparing a solution comprising at least one protein having a solubility in water superior or equal to about 10 mg/mL and at least one salt having solubility in water superior or equal to about 500 mg/mL, and
b) evaporating the solution obtained in step a) as is, as a foam obtained by foaming the solution obtained in step a), or as a mixture thereof, at a temperature comprised of from 4 to 50° C. in atmospheric pressure or at lower temperatures under vacuum or at a pressure lower than atmospheric pressure, until the formation of two non-miscible phases or until obtaining a substantially dry solid,
thereby obtaining a biomaterial.

2. The process according to claim 1, wherein said at least one salt is NaBr, NaI, KI, CaCl₂), MgCl2, KC2H3O2 and/or NH4HCO2.

3. The process according to claim 1, wherein said dry solid obtained in step b) is washed until elimination of at least 90% wt of said at least one salt.

4. The process according claim 1, wherein said at least one protein is a serum protein.

5. The process according to claim 4, wherein said serum protein is an albumin and the albumin is a human serum albumin, bovine serum albumin, porcine serum albumin, ovalbumin, vegetal albumin, or recombinant albumin.

6. The process according to claim 1, wherein at least one additive is added during step a) and/or step b).

7. The process according to claim 6, wherein said at least one additive is selected from polymers, non-ionic amino acids, and/or particles.

8. The process according to claim 1, wherein said at least one protein and said at least one salt are in a molar ratio (salt/protein) comprised between 100 and 3000.

9. A biomaterial obtainable by the process according to claim 1.

10. The biomaterial according to claim 9, wherein the percentage of secondary structures of said protein is at least the same as in corresponding native protein, when measured by IR analysis or Small angle X-ray scattering.

11. The biomaterial according to claim 9, wherein it is associated with at least one active ingredient.

12. The biomaterial according to claim 9, wherein said at least one active ingredient is selected from anti-cancer substances, anti-inflammatory agents, immunosuppressants, immunomodulators, modulators of cell-extracellular matrix interaction including cell growth inhibitors, anticoagulants, antithrombotic agents, enzyme inhibitors, analgesic, antiproliferative agents, antimycotic substances, cytostatic substances, growth factors, enzymes, hormones, steroids, non-steroidal substances, and/or anti-histamines.

13. A system comprising the biomaterial according to claim 9 and tissue and/or cells.

14. A drug comprising the biomaterial of claim 9.

15. A method for replacing defective tissues in a subject in need thereof, comprising introducing the biomaterial of claim 9 into the subject.

16. The process according to claim 4, wherein the serum protein is albumin or globulin.

17. The process according to claim 7, wherein the polymers are non-charged, positively charged, negatively charged and/or zwitterionic polymers.

18. A drug release system comprising the biomaterial of claim 9.

19. An implantable medical device comprising the biomaterial of claim 9.
